(19) United States
(12) Patent Application Publication
(10) Pub. No.: US 2015/0202220 A1
(43) Pub. Date: Jul. 23, 2015

(54) AGENTS, METHODS, AND DEVICES FOR AFFECTING NERVE FUNCTION

(71) Applicant: Northwind Medical, Inc., San Jose, CA (US)

(72) Inventors: Emily A. Stein, San Leandro, CA (US); Christina D. Swanson, Cambridge, MA (US); Michael A. Evans, Palo Alto, CA (US); Kondapavulur T. Venkateswara-Rao, San Jose, CA (US)

(73) Assignee: Northwind Medical, Inc., San Jose, CA (US)

(21) Appl. No.: 14/395,485

(22) PCT Filed: Oct. 25, 2012
(86) PCT No.: PCT/US2012/062006

§ 371 Date: Oct. 18, 2014

Related U.S. Application Data
(60) Provisional application No. 61/551,921, filed on Oct. 26, 2011.

(51) Int. Cl. A61K 31/7048; A61K 45/06; A61K 31/401; A61M 25/00; A61K 31/4166; A61K 31/55; A61K 38/17; A61M 25/10; A61K 9/00; A61K 31/405

(52) U.S. Cl. CPC A61K 31/7048; A61K 9/0012; A61K 45/06; A61K 31/401; A61K 31/405; A61K 31/4166; A61K 31/55; A61K 38/1787; A61M 25/10; A61M 25/0084; A61M 2025/1093; A61M 2025/0086

(57) ABSTRACT

Agents, methods, and devices for affecting nerve function are described. One embodiment of an agent includes a cardiac glycoside, an ACE inhibitor, and an NSAID. The agent may be delivered locally in a site-specific manner to a targeted nerve or portion of a nerve. For example, the agent may be delivered locally to the renal nerves to impair their function and treat hypertension. One embodiment of a delivery device includes one or more needle housings supported by a balloon. A delivery needle is slidably disposed within a needle lumen of each needle housing.

### CROSS-REFERENCE TO RELATED APPLICATION

This application claims the benefit of U.S. provisional patent application No. 61/551,921, filed Oct. 26, 2011, which is incorporated by reference in its entirety.

### BACKGROUND

Renal denervation involves denervating the renal nerves to treat hypertension. It has been found that sympathetic feedback from the kidneys is at least partially responsible for hypertension, and that the denervating of the renal nerves has the effect of lowering blood pressure.

One method of renal denervation involves the use of radiofrequency (RF) energy to ablate the renal nerves. An RF catheter is positioned inside the renal artery, and placed in contact with the wall of the renal artery, before RF energy is applied to the vascular tissue and renal nerves. The drawbacks of this approach include damage to the walls of the renal arteries and other surrounding tissue. Furthermore, the long-term effects of RF ablation are not well understood. For example, the response of the body to tissue killed by RF ablation may cause an undesirable necrosis or “dirty” response, versus an apoptosis response, which is a programmed, quiet cell death that triggers a phagocyte cleanup. Lastly, the destruction of the renal nerves by RF ablation is not a well-controlled (an all-or-none) process, and does not readily lend itself to adjustment in terms of specifically targeting nerve cells and limiting the damage caused to neighboring cells.

Another method of renal denervation involves the use of agents such as guanethidine or botulinum toxin to denervate the renal nerves. A delivery catheter is positioned inside the renal artery, and a needle is passed through the wall of the renal artery, before the guanethidine or botulinum toxin is injected in or around the renal nerves. However, these agents act at the synapses of sympathetic nerves. Because the renal nerves are made up of long nerve cells which begin at or near the spinal cord, or at or near the renal plexus near the aortic ostia of renal arteries, and terminate inside the kidneys, accessing the synapses well inside the kidneys makes local delivery difficult. This requires the delivery of large volume of agents over extended distances inside the body, and increases the likelihood of exposing renal tissue, surrounding tissue, and the kidneys to these agents.

What is needed are agents which can affect the function of nerves, while reducing the likelihood of damage to surrounding vascular and kidney tissues. What is needed are agents which can impair the function of the renal nerves, while reducing the likelihood of damage to the renal arteries and other tissues in the vicinity, and reducing the likelihood of damage to the kidneys. What is needed are agents which can permanently prevent neuronal signal transmission and insulate the kidney from the sympathetic electrical activity to and from the kidney over long periods of time. What is also needed are agents which can be titrated to control the amount of nerve function that is affected. What is also needed are agents that are effective in small volumes and low concentrations on a portion of the nerve or nerve cell, with minimal spillover into the systemic circulation and without affecting the central nervous system (CNS).

What is also needed are devices which can deliver these agents locally in small volumes to nerves and nerve cells in a targeted, site-specific manner, so as to reduce damage to surrounding tissues and reduce the side effects associated with systemic administration.

### SUMMARY

A method for treating hypertension in a patient is described. The method comprises delivering a mixture of a cardiac glycoside, an ACE inhibitor, and an NSAID locally to a portion of a renal nerve in an amount sufficient to impair function of the renal nerve and lower a blood pressure of the patient.

Also described is a method for treating a disease condition of the autonomic nervous system in a patient. The method comprises delivering an agent to a portion of a targeted nerve in an amount sufficient to affect function of the targeted nerve and alleviate one or more symptoms of the disease condition in the patient.

### BRIEF DESCRIPTION OF THE DRAWINGS

FIG. 1A shows a nerve cell 100 of the peripheral nervous system.

FIG. 1B shows an enlarged view of the axon 130.

FIG. 1C shows an enlarged view of a synapse 300.

FIGS. 2A-2E show how a voltage potential is maintained across the cell membrane 150 by a sodium-potassium pump 210.

FIGS. 3A-3E show how an action potential is propagated along the axon 130 by the sodium channels 220 and the potassium channels 230.

FIGS. 4A-4D show how a neural signal is propagated across a synapse 300.

FIG. 5 shows how a cardiac glycoside may affect nerve function.

FIG. 6 shows how a calcium channel blocker may affect nerve function.

FIG. 7 shows how a sodium channel blocker may affect nerve function.

FIG. 8 shows how an angiotensin-converting enzyme (ACE) inhibitor may affect nerve function.

FIG. 9 shows how an antibiotic may affect nerve function.

FIG. 10 shows how an excess amount of an excitatory amino acid may affect nerve function.

FIG. 11 shows how a non-steroidal anti-inflammatory drug (NSAID) affect nerve function.

FIGS. 12A-12D show the results of several different agents on rat sciatic nerves.

FIGS. 13A-13B show histologies at 72 hours and 30 days from the hind leg of a rat injected with digoxin.

FIGS. 14A-14G show one embodiment of a delivery catheter 400.

FIGS. 15A-15D show one embodiment of a method for using delivery catheter 400.

FIGS. 16A-16H show another embodiment of a delivery device 500.

FIGS. 17A-17D show one embodiment of a method for using delivery device 500.

FIGS. 18A-18E show yet another embodiment of a delivery device 600.

FIGS. 19A-19E show one embodiment of a method for using delivery device 600.

### DETAILED DESCRIPTION

The sympathetic nervous system represents one of the electrical conduction systems of the body. With age and disease, this electrical conduction system degenerates. The degeneration of the sympathetic nervous system is often accompanied by inflammation, expressed as overactivity of signal transmission or firing by the nerve cells. The agents, devices, and methods described below seek to affect the function of nerve cells by reducing or impairing this overactivity to treat a wide range of attendant disease conditions such as hypertension, diabetes, atrial fibrillation, sleep apnea, chronic kidney disease, obesity, dementia, depression, and many others.

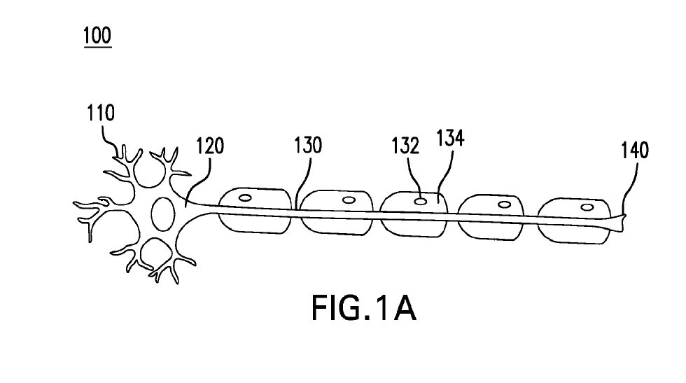

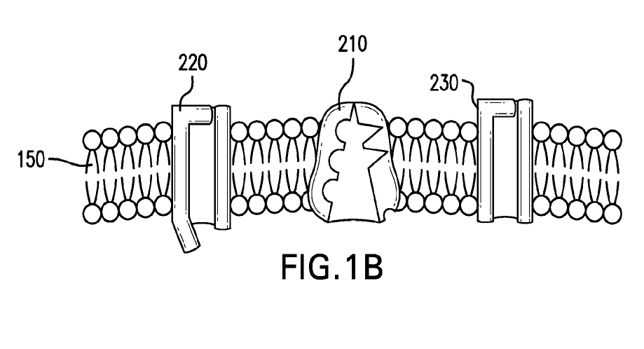

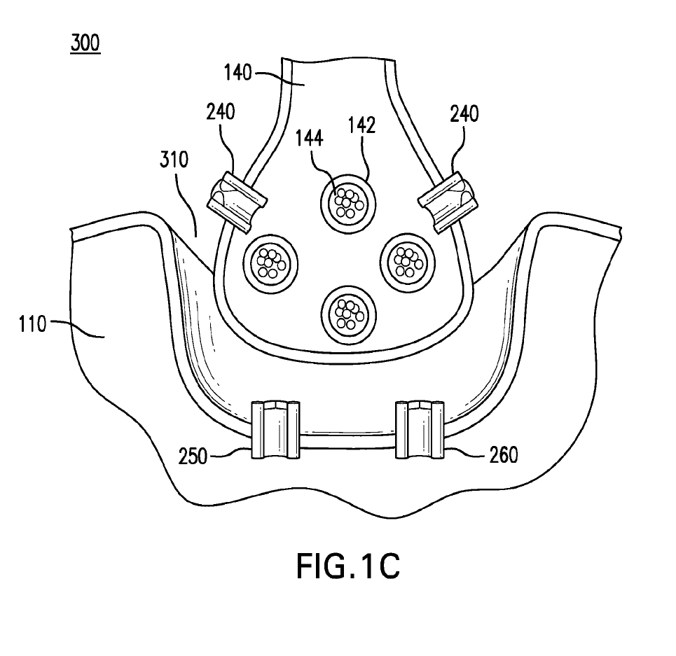

FIG. 1A shows a nerve cell 100 of the peripheral nervous system. The nerve cell 100 includes dendrites 110, a body 120, and an axon 130. The branches of the dendrites 110 receive from neural signals from other nerve cells and converge at the body 120. From the body 120, the axon 130 extends away and ends in axon terminals 140. An axon terminal 140 transmits neural signals to a dendrite of another nerve cell.

A nerve bundle is made up of a multiple of nerve cells. The individual nerve cells in a nerve bundle can perform different functions, depending on how the nerve cell is terminated. These functions include sensory, motor, pressure, and other functions.

The renal nerves may include nerve cells having axons of 5 to 25 cm or more in length, extending from the spinal cord to the kidney.

FIG. 1B shows an enlarged view of the axon 130, showing a cell membrane 150. The cell membrane 150 is embedded with sodium-potassium pumps 210, sodium channels 220, and potassium channels 230. The sodium-potassium pumps 210 maintain a voltage potential across the cell membrane 150. The sodium channels 220 and the potassium channels 230 propagate an action potential along the axon 130.

FIG. 1C shows an enlarged view of a synapse 300. An axon terminal 140 of a presynaptic nerve cell and a dendrite 110 of a postsynaptic nerve cell are separated by a synaptic cleft 310. The axon terminal 140 includes calcium channels 240 embedded in the cell membrane 150. The axon terminal also includes vesicles 142 containing neurotransmitters 144. The dendrite 110 of the postsynaptic nerve cell includes ligand-gated sodium channels 250 and ligand-gated calcium channels 260 which are activated by the neurotransmitters 144.

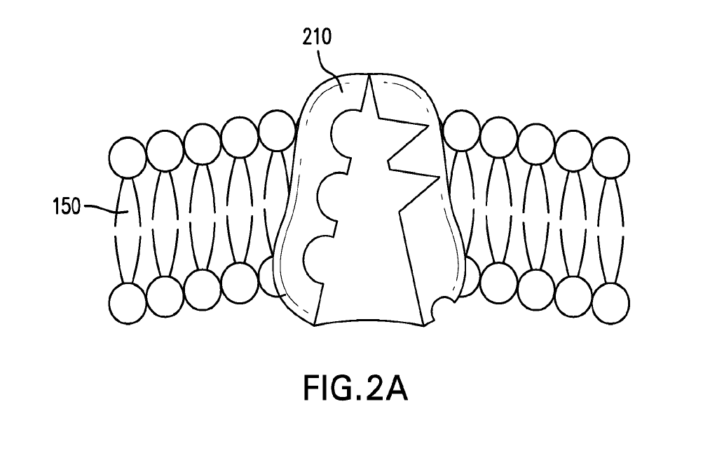

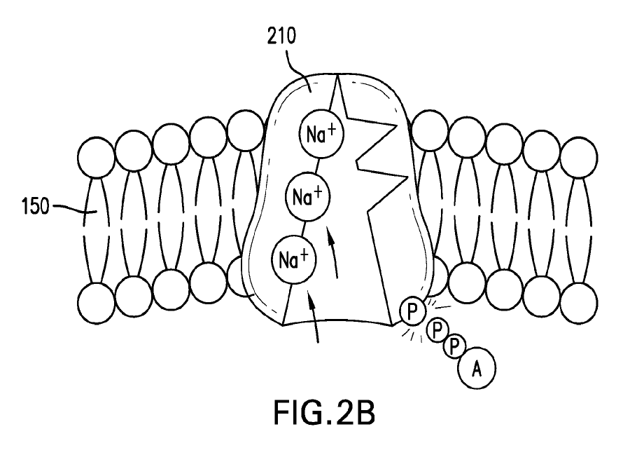

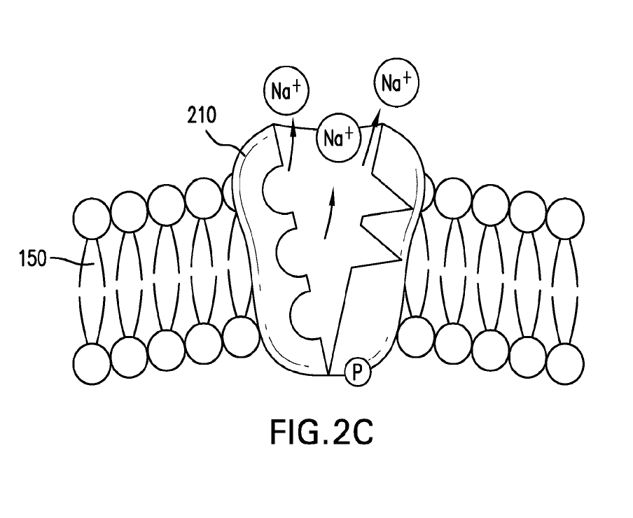

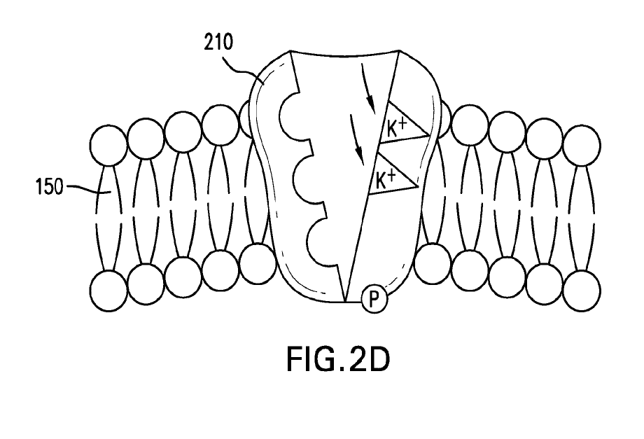

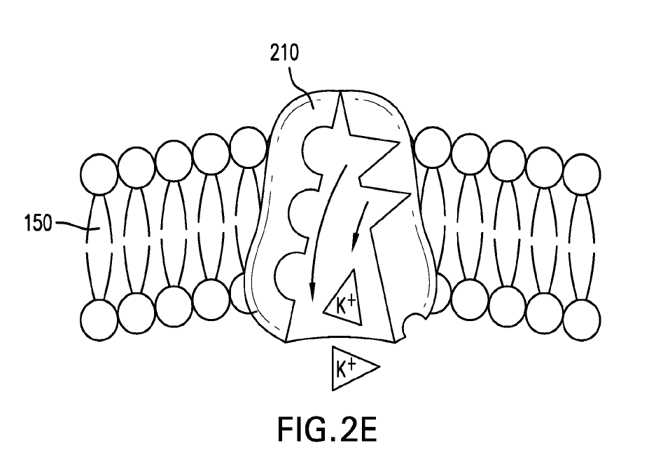

FIGS. 2A-2E show how a voltage potential is maintained across the cell membrane 150 by a sodium-potassium pump (Na+/K+-ATPase) 210. FIG. 2A shows a sodium-potassium pump 210 embedded in the cell membrane 150. FIG. 2B shows sodium ions (Na+) and an ATP molecule binding to the sodium-potassium pump 210 on the inside of the cell membrane 150. FIG. 2C shows the adenosine triphosphate (ATP) molecule being broken down into adenosine diphosphate (ADP), and the sodium-potassium pump 210 changing shape and transporting the sodium ions (Na+) to the outside of the cell membrane 150. FIG. 2D shows potassium ions (K+) binding to the sodium-potassium pump 210 on the outside of the cell membrane 150. FIG. 2E shows the phosphate molecule being released, and the sodium-potassium pump 210 reverting to its original shape and transporting the potassium ions (K+) to the inside of the cell membrane 150.

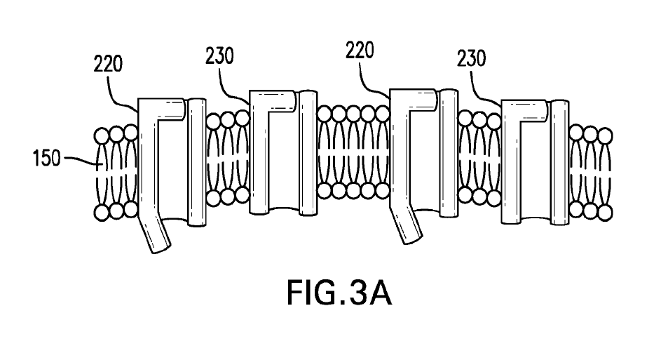

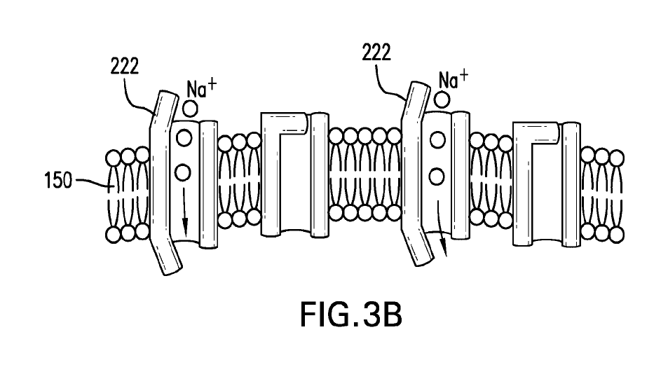

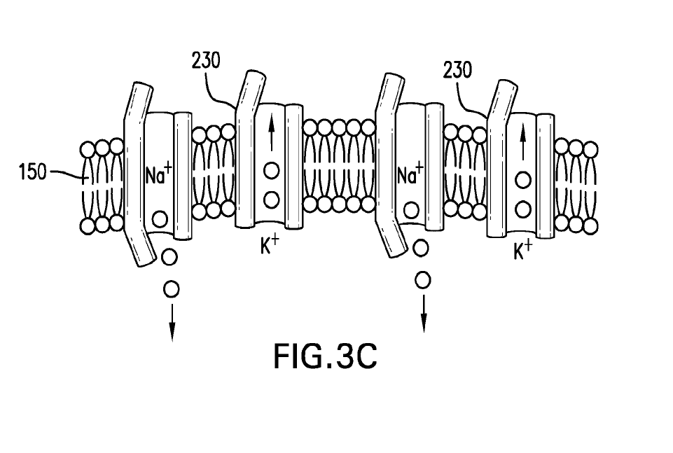

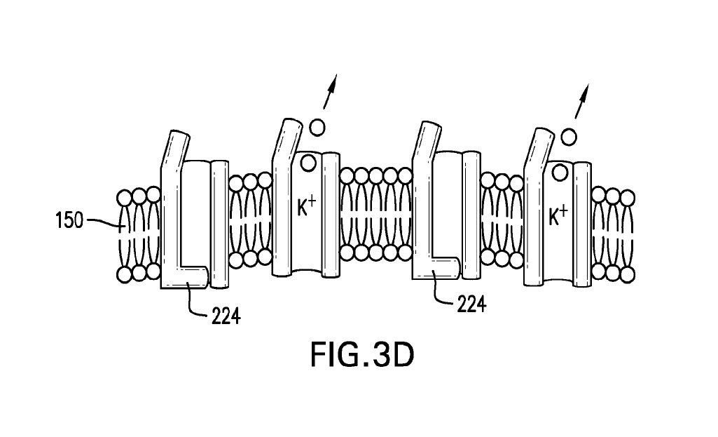

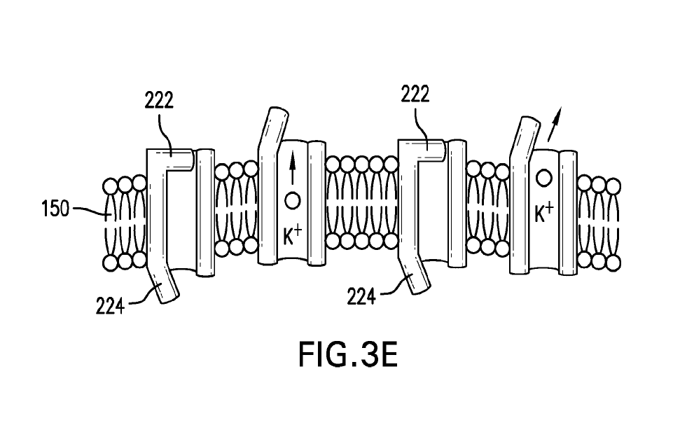

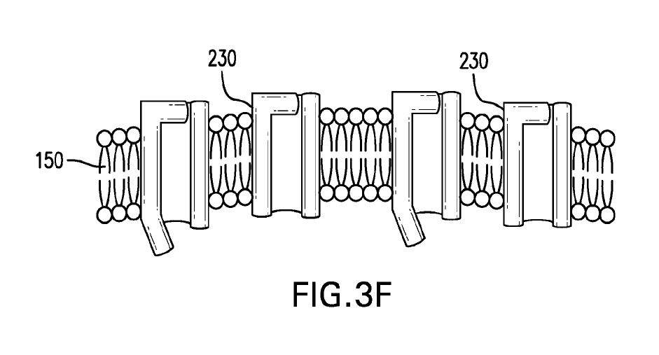

FIGS. 3A-3E show how an action potential is propagated along the axon 130 by the sodium channels 220 and the potassium channels 230. FIG. 3A shows sodium channels 220 and potassium channels 230 embedded in the cell membrane 150. FIG. 3B shows the arrival of an action potential, which opens activation gates 222 of the sodium channels 220, allowing the diffusion of sodium ions (Na+) into the inside of the cell membrane 150. FIG. 3C shows the action potential also opening the potassium channels 230, allowing the diffusion of potassium ions (K+) to the outside of the cell membrane 150. The combined effect of this is to depolarize the cell membrane 150, which propagates the action potential along the axon 130. FIG. 3D shows the inactivation gates 224 of the sodium channels 220 closed. FIG. 3E shows the activation gates 222 of the sodium channels 220 closed, and the inactivation gates 224 open. FIG. 3F shows the potassium channels 230 closed.

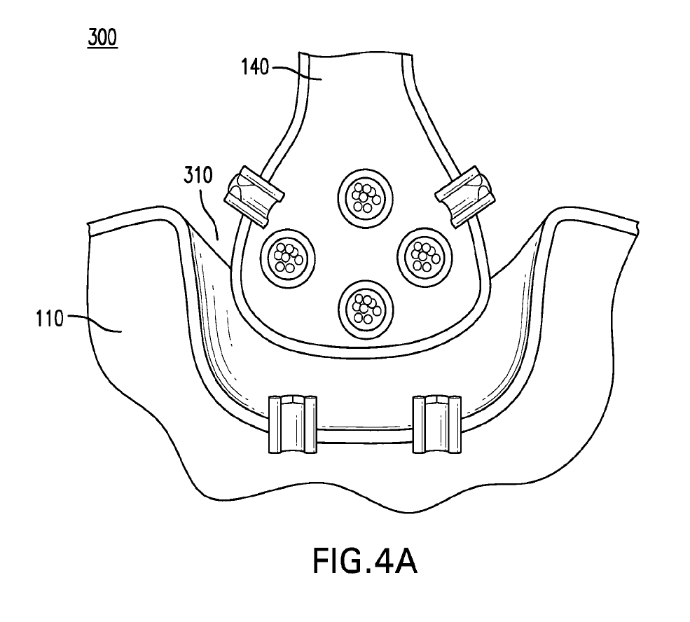

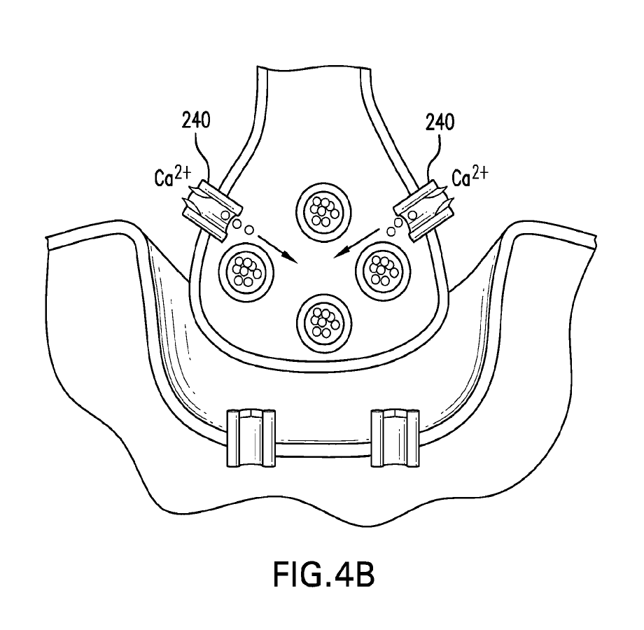

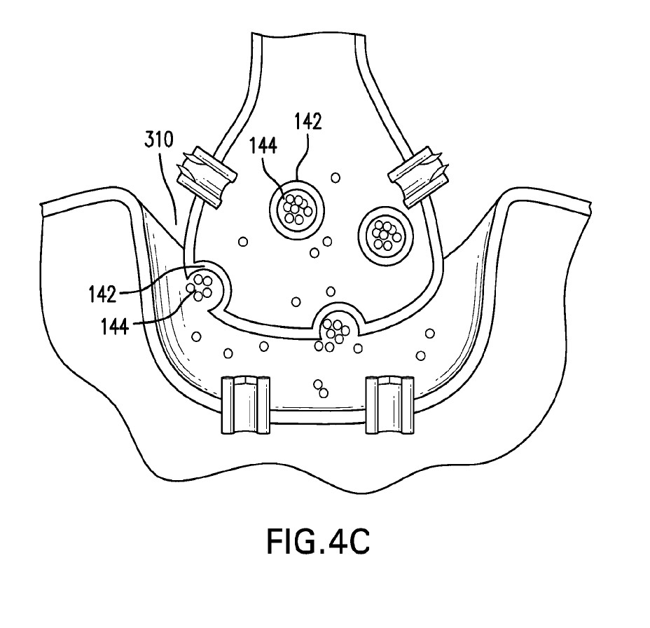

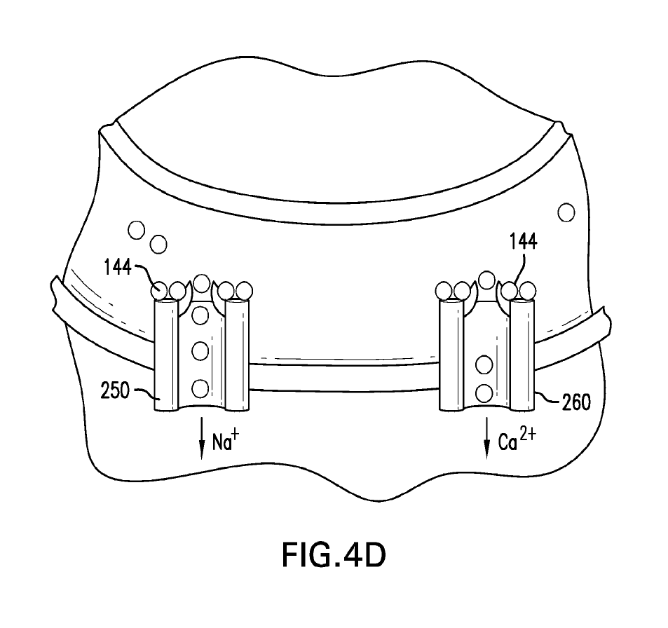

FIGS. 4A-4D show how a neural signal is propagated across a synapse 300. FIG. 4A shows an axon terminal 140 of a presynaptic nerve cell and a dendrite 110 of a postsynaptic nerve cell separated by the synaptic cleft 310. FIG. 4B shows the arrival of an action potential, which opens the calcium channels 240 and allows the diffusion of calcium ions (Ca2+) into the inside of the cell membrane 150. FIG. 4C shows the vesicles 142 releasing the neurotransmitters 144 into the synaptic cleft 310. FIG. 4D shows the neurotransmitters 144 binding to the ligand-gated sodium channels 250 and ligand-gated calcium channels 260, which opens them and allows the diffusion of sodium ions (Na+) and calcium ions (Ca2+) into the dendrite 110 to produce an action potential in the postsynaptic nerve cell.

Referring back to FIG. 1A, the axon 130 is surrounded by Schwann cells 132 which produce a myelin sheath 134 which covers the axon 130. The myelin sheath 134 is an insulator which serves to increase the speed of propagation of the action potential along the axon 130.

Several different classes of agents may be used to affect nerve function. These classes of agents act through different mechanisms.

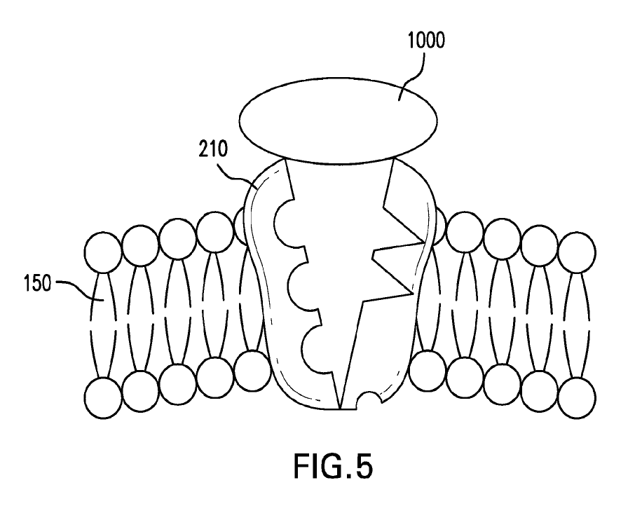

FIG. 5 shows how a cardiac glycoside may affect nerve function. Cardiac glycosides target sodium-potassium pumps 210. A cardiac glycoside molecule 1000 binds to the extracellular surface of a sodium-potassium pump 210. This inhibits the sodium-potassium pump 210, which reduces the transport of sodium ions out of the nerve cell 100. This increases the sodium ion concentration inside the nerve cell 100, which leads to apoptosis and impairs nerve function. Cardiac glycosides may also bind to organic anion transporters (OATs), which inhibits other membrane transport processes and leads to apoptosis. Cardiac glycosides include digoxin, proscillaridin, ouabain, digitoxin, bufalin, cymarin, oleandrin, and others.

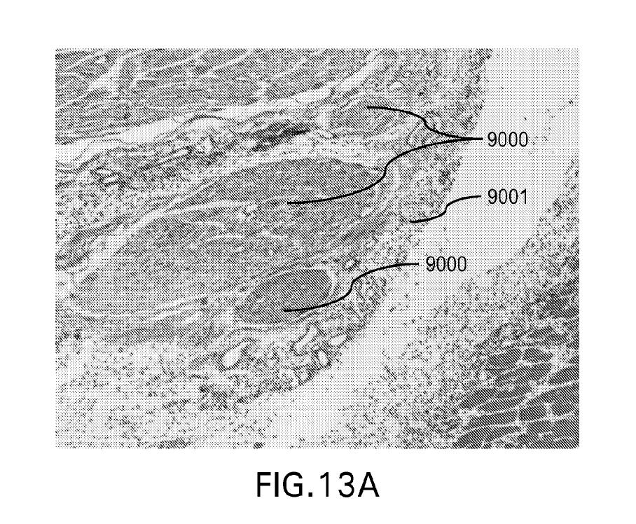

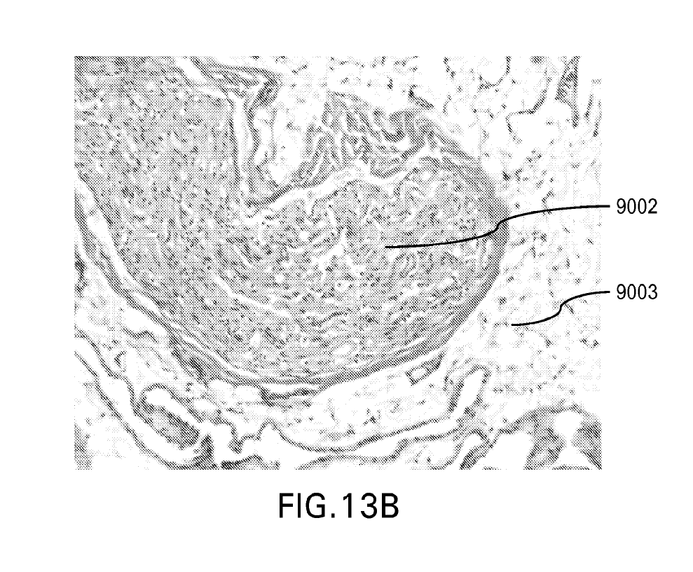

Cardiac glycosides may be delivered to a nerve in a targeted, site-specific manner, such as with the delivery devices described below and in FIGS. 13A-18F. They may target sodium-potassium pump along the long axonal segment of the nerve cell. This allows for a highly targeted and localized, site-specific effect by cardiac glycosides on a single nerve cell or a nerve cell bundle. This also allows for the use of very small volumes of agent delivered in a small, targeted area. This also allows the use of lower doses than when administered systemically, an advantage given the narrow therapeutic index of cardiac glycosides. This also avoids toxicity to other cells, given the amounts necessary to induce apoptosis, and given that many other types of cells other than nerve cells are also contain sodium-potassium pumps 210. This also avoids the need for the agents to be transported over large distances to reach the synaptic cleft, which may inhibit the transmission of catecholamines between neurons, as is the case with guanethidine, or the need to ablate large volumes of surrounding tissue to ablate nerves, as may happen with RF ablation.

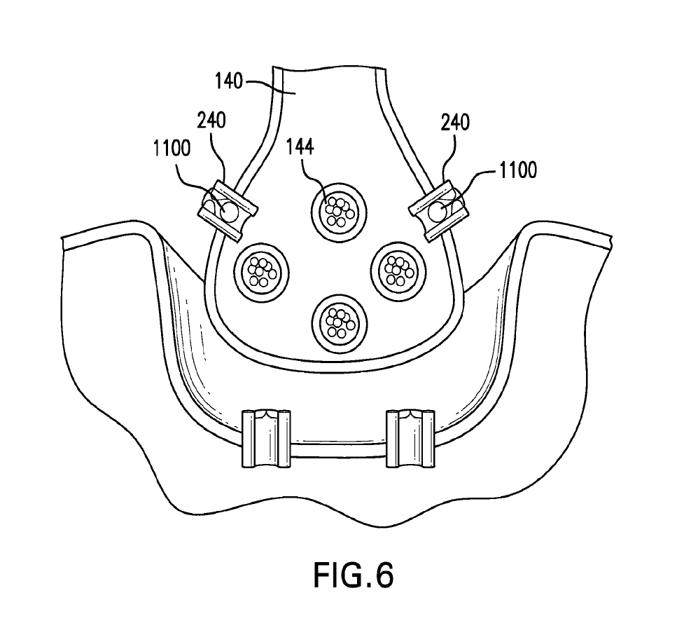

FIG. 6 shows how a calcium channel blocker may affect nerve function. Calcium channel blockers target calcium channels 240. A calcium channel blocker molecule 1100 binds to any one of several sites in a calcium channel 240, depending on the specific calcium channel blocker. This blocks the calcium channel 240, which inhibits the diffusion of calcium ions into the nerve cell 100 when an action potential is received. The lower calcium ion concentration inside the nerve cell 100 reduces the ability of the axon terminal 140 to release neurotransmitters 144 at the synapse 300, and thus impairs nerve function. Calcium channel blockers include amlodipine, aranidipine, azelnidipine, cilnidipine, felodipine and others.

Calcium channel blockers may be delivered to a nerve in a targeted, site-specific manner, such as with the delivery devices described below and in FIGS. 13A-18F. This allows the use of lower doses than when administered systemically. This also avoids impairing the function of cells other than the targeted nerve cells, given that many other types of cells other than nerve cells are also rich in calcium channels 240.

FIG. 7 shows how a sodium channel blocker may affect nerve function. Sodium channel blockers target sodium channels 220. A sodium channel blocker molecule 1200 binds to any one of several sites in a sodium channel 220, depending on the specific sodium channel blocker. This blocks the sodium channel 220, which inhibits the diffusion of sodium ions into the nerve cell 100 when an action potential is received. This inhibits the nerve from propagating action potentials and impairs nerve function. This effect is useful to inhibit high-frequency repetitive firing of action potentials caused by excessive stimulation. Sodium channel blockers include phenytoin, lithium chloride, carbamazepine, and others.

Sodium channel blockers may be delivered to a nerve in a targeted, site-specific manner, such as with the delivery devices described below and in FIGS. 13A-18F. This allows for delivery of low volumes of agent in small concentrations to the axonal segments of nerve cells, and effectively impairs nerve function with minimal damage to surrounding tissue or organs and limits the risk of the agents entering the systemic circulation. This also allows the use of lower doses than when administered systemically. This also avoids impairing the function of cells other than the targeted nerve cells, given that many other types of cells other than nerve cells are also rich in sodium channels 220.

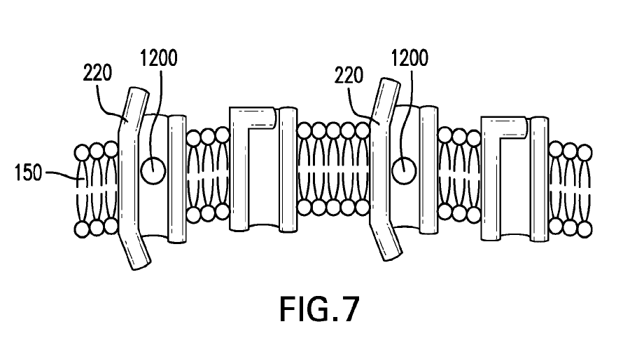

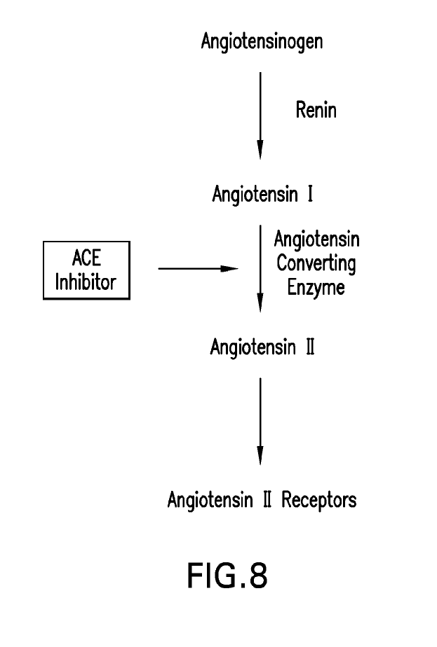

FIG. 8 shows how an angiotensin-converting enzyme (ACE) inhibitor may affect nerve function. ACE inhibitors target angiotensin-converting enzymes, disrupting the renin-angiotensin cycle. An ACE inhibitor inhibits ACE, which converts angiotensin I to angiotensin II, a more biologically active substrate for many cells including sympathetic nerves. ACE inhibition decreases angiotensin II production and thereby reduces nerve-specific production of norepinepherine. Blocking ACE by an ACE inhibitor not only reduces sympathetic nerve activity, it also decreases aldosterone release by the adrenal cortex. The combined effects result in the lowering of arteriolar resistance and renovascular resistance leading to increased excretion of sodium in the urine (natriuresis). ACE inhibitors include captopril, enalapril, lisinopril, ramipril, and others.

ACE inhibitors may be delivered to a nerve in a targeted, site-specific manner, such as with the delivery devices described below and in FIGS. 13A-18F. Site-specific administration of ACE inhibitors results in decreased local peripheral nerve activity.

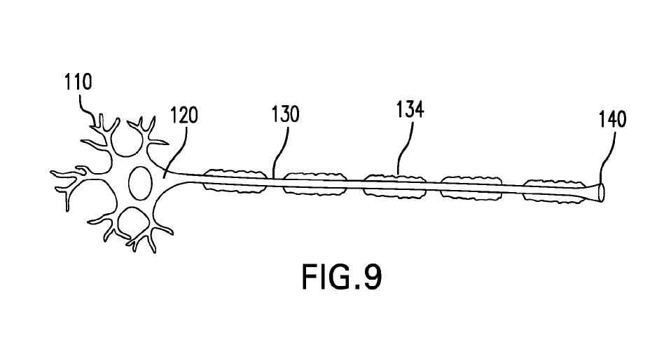

FIG. 9 shows how an antibiotic may affect nerve function. Antibiotics may cause RNA and thiamine antagonism. Antibiotics may also cause demyelination of the nerve cells, which interferes with the ability of the nerve cells to conduct signals. The fluoroquinolone class of antibiotics has been shown to cause irreversible peripheral neuropathy. Antibiotics include metronidozole, fluoroquinolones (such as ciprofloxacin, levofloxacin, moxifloxacin and others), chloramphenicol, chloriquine, clioquinol, dapsone, ethambutol, griseofulvin, isoniazid, linezolid, mefloquine, nitrofurantoin, podophyllin resin, suramin, and others.

Antibiotics may be delivered to a nerve in a targeted, site-specific manner, such as with the delivery devices described below and in FIGS. 13A-18F. This allows the use of lower doses than when administered systemically, an advantage given the effects of some of these antibiotics on the central nervous system. This also minimizes damage to other tissue in the vicinity of the targeted nerve.

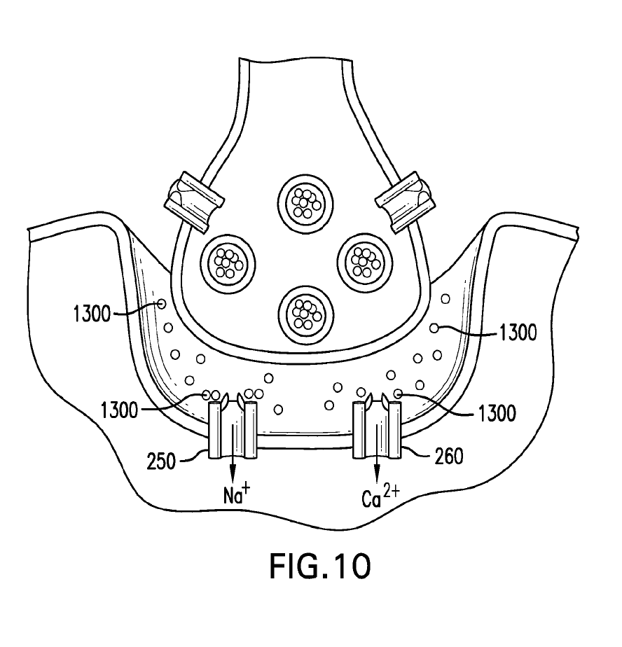

FIG. 10 shows how an excess amount of an excitatory amino acid may affect nerve function. Excitatory amino acids target neurotransmitter receptors in the postsynaptic nerve cell. An excess amount of an excitatory amino acid 1300 overactivates the neurotransmitter receptors of the sodium channels 250 and calcium channels 260, which leads to the uptake of high amounts of sodium and calcium ions in the postsynaptic nerve cell. These high sodium and calcium ion concentrations lead to destruction of cell components, apoptosis, and impaired nerve function. Excitatory amino acids include monosodium glutamate, domoic acid and others.

Excess amounts of excitatory amino acids may be delivered to a nerve in a targeted, site-specific manner, such as with the delivery devices described below and in FIGS. 13A-18F. This allows the use of lower doses than when administered systemically. This also avoids impairing the function of cells other than nerve cells, given that many other types of cells other than nerve cells are also rich in calcium channels 240.

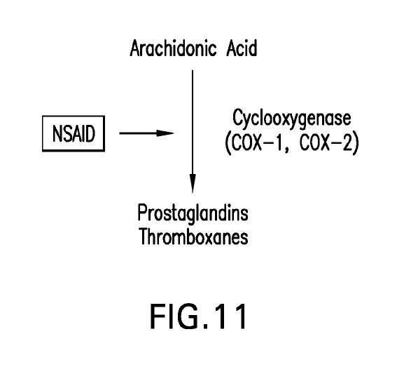

FIG. 11 shows how a non-steroidal anti-inflammatory drug (NSAID) may affect nerve function. NSAIDs target the cyclooxygenase (COX) enzyme. An NSAID blocks the COX-1 and COX-2 enzymes, which suppresses production of prostaglandins and thromboxanes and reduces synaptic signaling. Additionally, a subclass of prostaglandins are involved in healing and the administration of prostaglandin E2 enhances healing. Like other analgesics, NSAIDs can act in various ways on the peripheral and central nervous systems. NSAIDs include indomethacin, aspirin, ibuprofen, naproxen, celecoxib, and others.

NSAIDs may be delivered to a nerve in a targeted, site-specific manner, such as with the delivery devices described below and in FIGS. 13A-18F. This is advantageous over systemic administration because of adverse drug reactions (ADRs) to NSAIDs in the kidneys. Blocking prostaglandin production in the kidneys is undesirable, as prostaglandins are essential in maintaining normal glomerular perfusion and glomerular filtration rate.

Agents for affecting nerve function may include agents having a single component, as well as agents having a combination of two or more components. There are several advantages to the use of combinatorial agents to affect the function of nerve cells. First, different agents act on different targets on the nerve cells and improve the efficacy of action. Second, there may be synergistic effects in which a first agent prevents firing (release of neurotransmitters, polarization, and/or opening of channels) of the nerve cells and a second agent prevents repolarization. Third, the synergistic effect of two or more agents allows the concentration of the components within the formulation to be lowered compared to use of a single agent, while still achieving a desired efficacy.

A first embodiment of an agent for affecting nerve function includes: (1) digoxin (a cardiac glycoside), (2) captopril (an ACE inhibitor), and (3) indomethacin (an NSAID). The digoxin dose may be approximately 0.2-2.0 mg/kg. The captopril dose may be approximately 2-20 mg/kg. The indomethacin dose may be approximately 0.2-20 mg/kg.

Digoxin is FDA-approved, comes in injectable formulations, and is available as a generic. The pharmacokinetic and pharmacodynamic properties of digoxin are desirable for affecting nerve function. Digoxin is extremely hydrophobic and the high lipid content surrounding nerves and nerve bundles allows digoxin to penetrate the outer lipid-rich sheath. Digoxin has a half-life of 36-48 hours in healthy individuals and is excreted by the renals, which reduce the risk of diffusion-related effects on sites outside of the zone of administration. Other cardiac glycosides with lipophilic profiles include bufalin, ouabain, and others.

Captopril is FDA-approved, is available as a generic, has a streamlined synthesis, comes in injectable formulations, has a well-established safety profile, and has a well-established dosing regimen. Captopril is excreted by the renals with a short half-life of 1.9 hours.

Indomethacin is FDA-approved, comes in injectable formulations, and is available as a generic. Indomethacin has a half-life of 4.5 hours and the majority of the agent is excreted by the renals.

A second embodiment of an agent for affecting nerve function includes: (1) digoxin (a cardiac glycoside), and (2) indomethacin (an NSAID).

A third embodiment of an agent for affecting nerve function includes: (1) digoxin (a cardiac glycoside), and (2) lithium chloride (a sodium channel blocker).

A fourth embodiment of an agent for affecting nerve function includes: (1) ouabain (a cardiac glycoside), (2) carbamazepine (a sodium channel blocker), and (3) captopril (an ACE inhibitor).

A fifth embodiment of an agent for affecting nerve function includes: (1) metrodinazole (an antibiotic), (2) captopril (an ACE inhibitor), and (3) indomethacin (an NSAID).

A sixth embodiment of an agent for affecting nerve function includes: (1) digoxin (a cardiac glycoside), (2) lithium chloride (a sodium channel blocker), and (3) amlodipine (a calcium channel blocker).

### Example 1

The efficacy of various agents in affecting nerve function was evaluated using a rat sciatic nerve block model. Rat groups were injected with 0.3 cc agent formulations in the left leg near the sciatic notch. The rat groups, agents, and doses are listed in the table below:

| GROUP | AGENT | DOSE (mg/kg) |
|---|---|---|
| 1 | Ethanol | 100% |
| 2 | Guanethidine | 5.77 |
| 3 | Digoxin | 1.06 |
| 4 | Carbamazepine | 1.44 |
| 5 | Phenytoin | 3.82 |
| 6 | Digoxin + carbamazepine | 0.27, 0.36 |
| 7 | Digoxin + captopril + indomethacin | 0.27, 5.88, 0.22 |

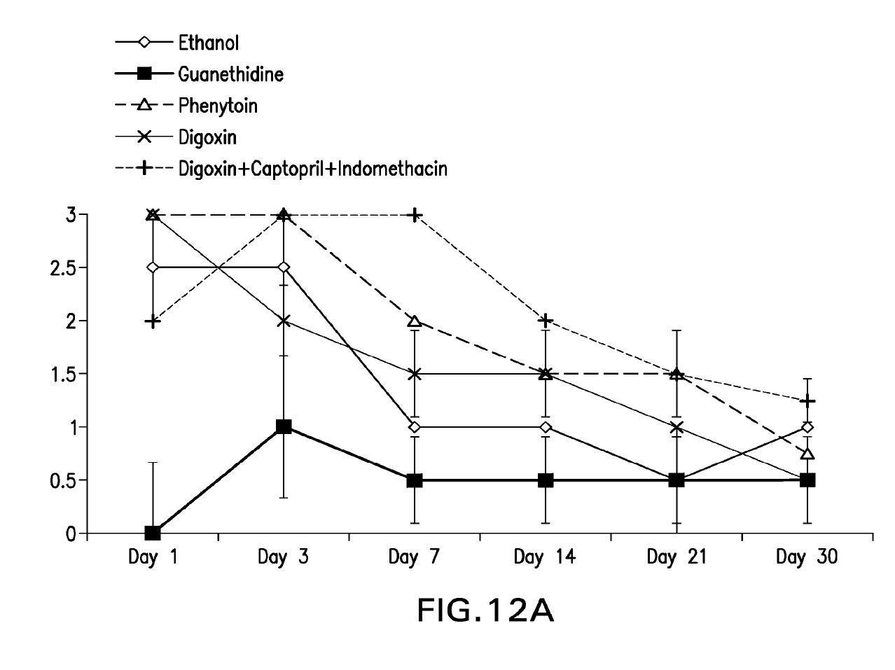

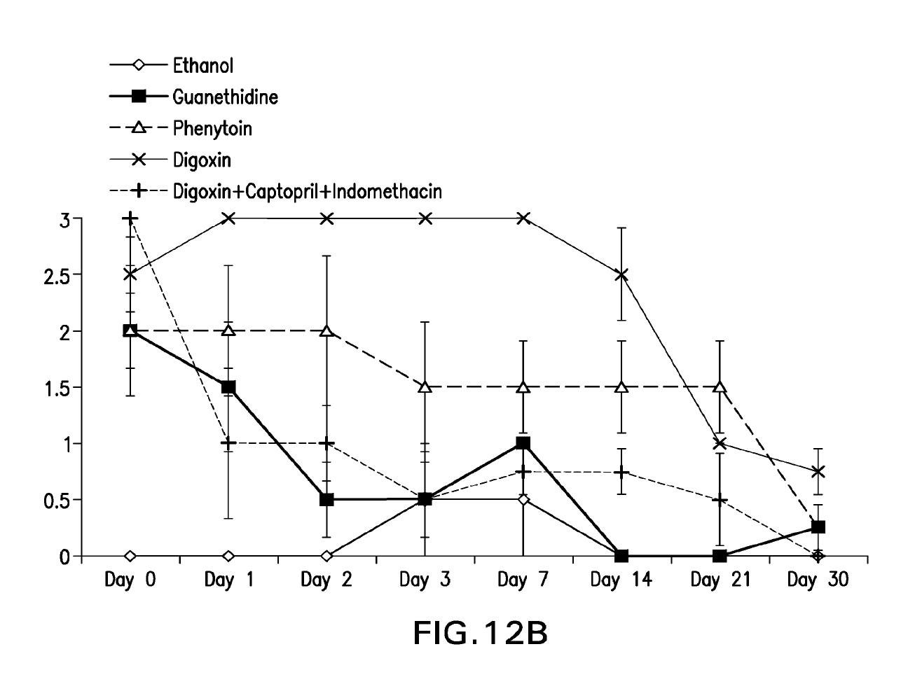

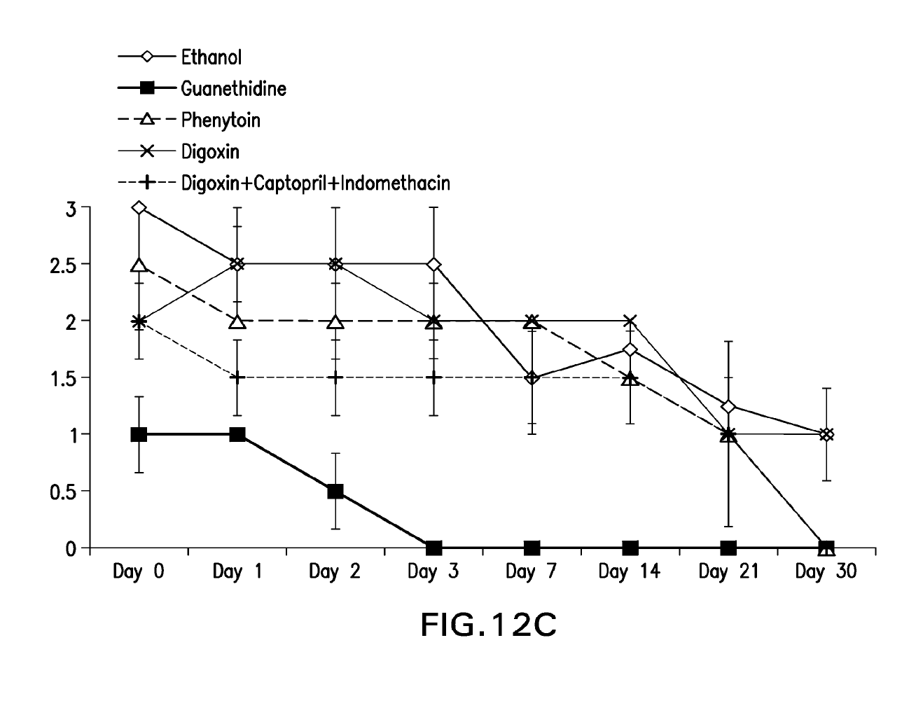

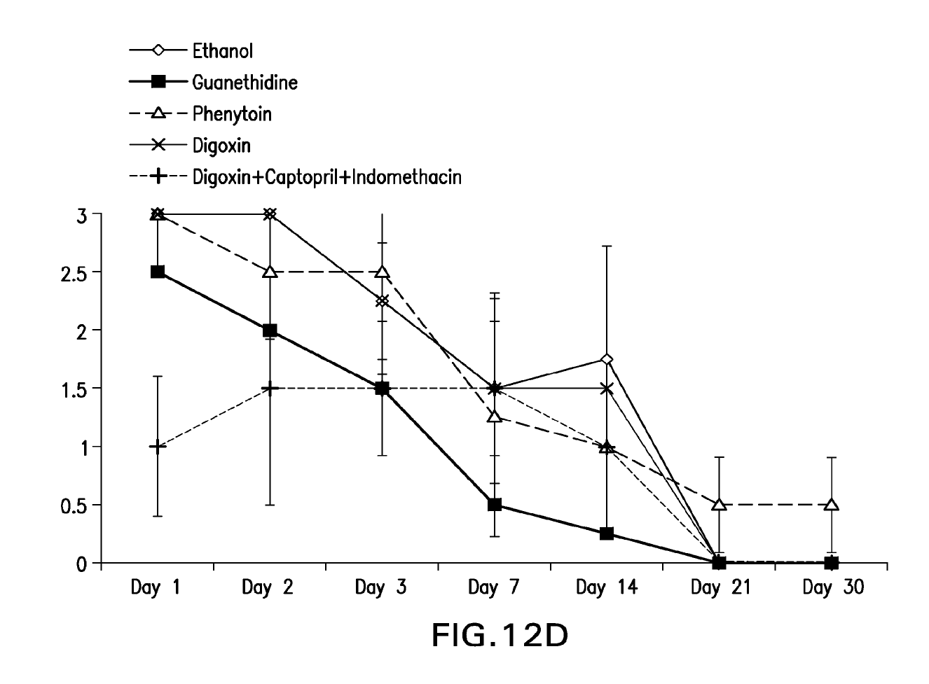

FIGS. 12A-12D show the results of the different agents on the rat leg muscles. The effect of the agents was measured based on four tests: (1) nerve conductance, (2) sensory ability, (3) motor function, and (4) pressure exerted.

FIG. 12A shows the results of the nerve conductance test. The nerve conductance test evaluates the ability of electrical current to travel from one electrode, down the sciatic nerve and to a second electrode to form a complete electrical circuit. Nerve conductance was evaluated at 2 frequencies (1-10 Hz to stimulate leg twitch and 50-100 Hz to stimulate leg tetanus). Impairment in nerve conductance was evaluated at 1, 2, 3, 7, 14, 21, and 30 days post-injection of agent. The y-axis scale represents the severity of block (on a scale of 0-3, with 0=no block, 1=slight block, 2=moderate block, 3=severe block).

FIG. 12B shows the results of the sensory ability test. The sensory ability test evaluates sensory nerve function. Needle-nosed forceps were used to pinch the footpad of rat hindlimbs to test ability of sensory nociception. Vocal responses or mechanical withdraw of the foot from the forceps were monitored as pressure increased. Rats were assessed at 1, 2, 3, 7, 14, 21, and 30 days. The y-axis scale represents the severity of sensory nociception block (on a scale of 0-3, with 0=no block, 1=slight block, 2=moderate block, 3=severe block).

FIG. 12C shows the results of the motor function test. The motor function test evaluated the ability of rats to step up, walk, and coordinate their hindlimbs. The measurements were made at 1, 2, 3, 7, 14, 21, and 30 days. The y-axis scale represents the severity of neuromuscular block (on a scale of 0-3, with 0=no block, 1=slight block, 2=moderate block, 3=severe block).

FIG. 12D shows the results of the pressure exerted test. The pressure exerted test evaluated the ability of rats to apply pressure or bear weight on a flat surface which was measured by a digital weighing scale. The measurements were made at 1, 2, 3, 7, 14, 21, and 30 days. The y-axis scale represents the impairment in the ability to bear weight (on a scale of 0-3, with 0=no impairment, 1=slight impairment, 2=moderate impairment, 3=severe impairment).

These data suggest cardiac glycosides, either alone or in combination with an ACE inhibitor and NSAID, outperform guanethidine in the ability to affect peripheral nerve function. Additionally, cardiac glycosides outperform other tested agents, including ethanol, in the ability to impair sensory nociception.

A lower amount of digoxin is needed to affect nerve function when used in conjunction with captopril and indomethacin than when used alone. This synergistic effect may be due to the effect of the captopril and the indomethacin within the same nerve cell, on the neighboring cells, or in the local micro-environment surrounding the nerve cells, nerve cell bundle, or nerve cell junction. For example, co-administration of captopril had the effect of inhibiting angiotensin II production and reducing nerve stimulation, resulting in decreased nerve activity (e.g., norepinephrine production) in the injected tissue. Additionally, co-administration of indomethacin blocked COX-2 activity and prostaglandin production, and therefore decreased healing, which prolonged the effects of digoxin and captopril.

Separate components of an agent for affecting nerve function may be administered using different routes. For digoxin, captopril, and indomethacin, the digoxin may be administered locally in a site-specific manner, while the captopril and the indomethacin may be administered orally or intravenously. The synergistic effects are still seen, as the combined effects of three separate mechanisms affecting nerve function appear to require smaller doses or local concentrations of each component.

FIG. 13A shows histology at 72 hours from the hind leg of a rat injected with digoxin. The nerve bundles 9000 contain nerve axons showing signs of edema and axonal degeneration. The nerve bundles are surrounded by perineuritis 9001.

FIG. 13B shows histology at 30 days from the hind leg of a rat injected with digoxin. The nerve bundles 9002 contain degenerated nerves. The absence of inflammatory foci surrounding the degenerative nerve bundles is also noted 9003.

The following table is a summary of the effects of three different agents on the nerve cells:

| | Time | | Sciatic Nerve | Inflammatory |
|---|---|---|---|---|
| Agent | Point | | Pathology Report | Condition |
| Phenytoin | 72 | hrs | Normal | Normal |
| | 30 | days | Normal | Perineuritis |
| Digoxin | 72 | hrs | Normal | Perineuritis |
| | 30 | days | Degenerative with some | No |
| | | | edema; endoneurium is | inflammation |
| | | | absent; nerve is fragmented; | |
| | | | axonal degeneration is present | |
| Digoxin + | 72 | hrs | Nerve degeneration | No |
| captopril + | | | with edema | inflammation |
| indometh- | 30 | days | Axonal degeneration | No |
| acin | | | with some swelling; | inflammation |
| | | | no hypercellularity | |

For local delivery performed under fluoroscopy, small amounts of radioopaque contrast agents (commercially available agents like Omnipaque and others) may be included in a formulation without compromising its efficacy. These contrast agents provide visual confirmation that the agent is being delivered to the target location during the clinical procedure. Both ionic and non-ionic contrast agents can be used. Examples include diatrizoate (Hypaque 50), metrizoate (Isopaque 370), ioxaglate (Hexabrix), iopamidol (Isovue 370), iohexol (Omnipaque 350), ioxilan (Oxilan 350), iopromide (Ultravist 370), and iodixanol (Visipaque 320).

Local delivery of agents to affect nerve function may not be permanent, lasting from a few months to a few years. The sympathetic nervous system may return to its degenerated, overactive condition as the nerve cells regrow and transmit signals to and from the kidneys. If an extended effect is desired, agents may be included that may prevent nerve cell regrowth locally without causing detrimental effects to the central nervous system or surrounding tissue to permanently impair or affect nerve function and prevent nerve overactivity. These agents include a variety of nerve growth inhibitors, which may be used in a time-release formulation.

Nerve growth inhibitors prevent regrowth of the nerve after nerve cell injury or nerve cell death. Nerve growth inhibitors may prolong the effect on nerve function from months to years, or even make permanent the effect on nerve function.

A nerve growth inhibitor may be a single agent, or include two or more agents. A nerve growth inhibitor may include a small molecule inhibitor, a kinase inhibitor, a neutralizing or blocking antibody, a myelin-derived molecule, a sulfate proteoglycan, and/or extracellular matrix components.

Small molecule inhibitors may include, but are not limited to, cyclic-adenosine analogs and molecules targeting enzymes including Arginase I, Chondroitinase ABC, β-secretase BACE1, urokinase-type plasminogen activator, and tissue-type plasminogen activator. Inhibitors of arginase include, but are not limited to, N-hydroxy-L-arginine and 2(S)-amino-6-boronohexonic acid. β-secretase inhibitors include, but are not limited to, N-Benzyloxycarbonyl-Val-Leu-leucinal, H-Glu-Val-Asn-Statine-Val-Ala-Glu-Phe-NH₂, H-Lys-Thr-Glu-Glu-Ile-Ser-Glu-Val-Asn-Stat-Val-Ala-Glu-Phe-OH. Inhibitors of urokinase-type and tissue-type plasminogen activators include, but are not limited to, serpin E1, Tiplaxtinin, and plasminogen activator inhibitor-2.

Kinase inhibitors may target, but are not limited to targeting, Protein Kinase A, PI 3 Kinase, ErbB receptors, Trk receptors, Jaks/STATs, and fibroblast growth factor receptors. Kinase inhibitors may include, but are not limited to, staurosporine, H 89 dihydrochloride, cAMPS-Rp, triethylammonium salt, KT 5720, wortmannin, LY294002, IC486068, IC87114, GDC-0941, Gefitinib, Erlotinib, Lapatinib, AZ623, K252a, KT-5555, Cyclotraxin-B, Lestaurtinib, Tofacitinib, Ruxolitinib, SB1518, CYT387, LY3009104, TG101348, WP-1034, PD173074, and SPRY4.

Neutralizing or blocking antibodies may target, but are not limited to targeting, kinases, enzymes, integrins, neuregulins, cyclin D1, CD44, galanin, dystroglycan, repulsive guidance molecule, neurotrophic factors, cytokines, and chemokines Targeted neurotrophic factors may include, but are not limited to, nerve growth factor, neurotrophin 3, brain-derived neurotrophic factor, and glial-cell-line derived neurotrophic factor. Targeted cytokines and chemokines may include, but are not limited to, interleukin-6, leukemia inhibitor factor, transforming growth factor β1, and monocyte-chemotactic protein 1.

Myelin-derived molecules may include, but are not limited to, myelin-associated glycoprotein, oligodendrocyte myelin glycoprotein, Nogo-A/B/C, Semaphorin 4D, Semaphorin 3A, and ephrin-B3.

Sulfate proteoglycans may include, but are not limited to, keratin sulfate proteoglycans and chondroitin sulfate proteoglycans such as neurocan, brevican, versican, phosphacan, aggrecan, and NG2.

Extracellular matrix components may include, but are not limited to, all known isoforms of laminin, fibrinogen, fibrin, and fibronectin.

Fibronectin binds to integrins such as alpha5beta1 on Schwann cells and neurons. Schwann cells adhere to fibronectin in order to migrate, and fibronectin acts as chemo-attractant and mitogen to these cells. Fibronectin aids the adhesion and outgrowth of regenerating axons. Agents which target fibronectin to impair nerve regrowth may thus include (1) isoforms of fibronectin that antagonize, rather than promote, integrin signaling, (2) blocking/neutralizing antibodies against certain fibronectin isoforms that promote integrin signaling, and/or (3) blocking/neutralizing antibodies that reduce fibronectin/integrin binding, integrin internalization or integrin grouping. One example of a humanized monoclonal antibody targeting fibronectin is Radretumab.

Laminins mediate the adhesion of neurons and Schwann cells to the extracellular matrix acting as a guide and “go” signal for regrowth. Laminin chains such as alpha2, alpha4, beta1 and gamma1 are upregulated following peripheral nerve injury and signal to neurons and Schwann cells through beta1 integrins such as alpha1beta1, alpha3beta1, alpha6beta1 and alpha7beta1 integrins. Agents which target laminins to impair nerve regrowth may thus include (1) antibodies that neutralize the effects of laminins, (2) laminin isoforms that antagonize rather than promote axon regrowth, and/or (3) blocking/neutralizing antibodies that reduce laminin/integrin binding, integrin internalization, or integrin grouping.

Collagen and fibrin promote nerve repair of a gap when added to the gap at low concentration, oriented in a longitudinal manner. However, fibrin (and perhaps collagen) may hinder nerve regeneration in some situations. First, unorganized fibrinogen in gel may retard nerve regeneration by confusing the growth pathways. Second, mice deficient in fibrinolytic enzymes such as tissue plasminogen activator or plasminogen have exacerbated injuries after sciatic nerve crush. This is believed to be due to fibrin deposition as fibrin depletion rescued the mice. In vitro experiments showed that fibrin downregulated Schwann cell myelin production and kept them in a proliferating, nonmyelinating state. Thus, at least a few different agents may be used to impair nerve regrowth. First, collagen or fibrinogen or the combination may be added at high concentration, in an unorganized state, via a gel injection at the site of injury. Second, small molecule inhibitors or neutralizing antibodies against tissue plasminogen activator or plasminogen may be used. Third, fibrin deposition may be mimicked by addition of peptides with the heterodimeric integrin receptor binding sequence arginine-glycin-asparagin.

Neurotrophic factors promote the growth of neurons. These include Nerve Growth Factor, Neurotrophin 3, Brain-derived neurotrophic factor. Agents which target neurotrophic factors to impair nerve regrowth may thus include neutralizing/blocking antibodies against neurotrophic factors or their respective receptors.

Glial growth factor (GGF) is produced by neurons during peripheral nerve regeneration, and stimulates the proliferation of Schwann cells. Agents which target GGF to impair nerve regrowth may thus include blocking/neutralizing antibodies against GGF.

Cyclic adenosine monophosphate (cAMP) is a second messenger that influences the growth state of the neuron. cAMP activates Protein Kinase A which induces the transcription of IL-6 and arginase I. Arginase I synthesizes polyamines which is considered one way that cAMP promotes neurite outgrowth. Knowledge of this pathway that promotes neurite outgrowth allows for identification of numerous targets for inhibiting neurite outgrowth. For instance, cAMP and Protein Kinase A may be targeted. Although the stereospecific cAMP phosphorothioate analog activates Protein Kinase A, other conformation such as the antagonistic Rp-cAMPs inhibit Protein Kinase A activity and may thus be used. Small molecules that inhibit Protein Kinase A or neutralizing/blocking antibodies that prevent cAMP from binding Protein Kinase A, or that prevent activation of Protein Kinase A via an alternative mechanism, may be used. Examples of inhibitors of Protein Kinase A include H 89 dihydrochloride, cAMPS-Rp, triethylammonium salt, and KT 5720. Further down the pathway, small molecule inhibitors of arginase I and polyamine synthesis may be used to reduce neurite outgrowth. Inhibitors of Arginase I may include but are not limited to, 2(S)-amino-6-boronohexonic acid and other boronic acid inhibitors.

Myelin-associated inhibitors are components of myelin expressed in the CNS by oligodendrocytes that impair neurite outgrowth in vitro and in vivo. Myelin-associated inhibitors include Nogo-A, myelin-associated glycoprotein (MAG), oligodendrocyte myelin glycoprotein (OMgp), ephrin-B3, and semaphorin 4D. NogoA, MAG and OMgp interact with Nogo-66 receptor 1 and the paired immunoglobulin-like receptor B to limit axon growth. Furthermore, transgenic expression of Nogo C, an isoform on Nogo A, in Schwann cells delays peripheral nerve regeneration. Any of these may be used to impair nerve regrowth.

Chondroitin sulfate proteoglycans (CSPGs) are upregulated by reactive astrocytes in the glial scar following nerve injury. They include neurocan, versican, brevican, phosphacan, aggrecan and NG2. Interfering with CSPG function is known to promote nerve growth in the CNS. Thus, CSPGs may be used to reduce nerve regrowth.

Non-myelin derived axon regeneration inhibitors are found in the CNS, but not derived from myelin. They include repulsive guidance molecule (RGM) and semaphorin 3A. Antibodies or small molecule inhibitors targeting these molecules promote functional recovery following spinal cord injury in rats. Thus, these molecules may be used to reduce nerve regrowth. Furthermore, these molecules activate Rho A which activates ROCK2 kinase, indicating that small molecules or antibodies that activate ROCK2 may be used to reduce neurite outgrowth. Examples of ROCK2 inhibitors include Fasudil hydrochloride which inhibits cyclic nucleotide dependent- and Rho-kinases, HA 1100 hydrochloride which is a cell-permeable, Rho-kinase inhibitor, dihydrochloride which is a selective Rho-kinase (ROCK) inhibitor, and dihydrochloride which is a selective inhibitor of isoform p160ROCK.

Time-release formulations may include the use of microspheres made from biodegradable polymer matrices containing the agents, bioerodible matrices, and biodegradable hydrogels or fluids that have prolonged agent release rates and degradation profiles. The agent is released as the polymer degrades and non-toxic residues are removed from the body over a period of week to months. Useful polymers for the biodegradable controlled release microspheres for the prolonged administration of agents to a targeted site include polyanhydrides, polylactic acid-glycolic acid copolymers, and polyorthoesters. Polylactic acid, polyglycolic acid, and copolymers of lactic acid and glycolic acid are preferred. Other polymer matrices include polyethylene glycol hydrogels, chitin, and polycaprolactone copolymers

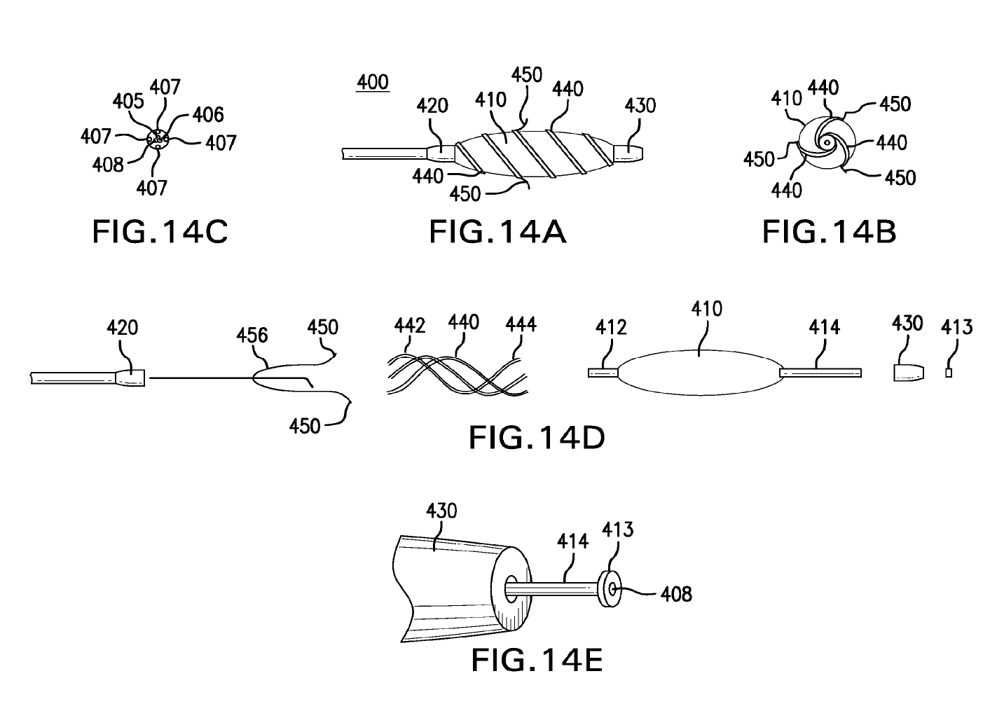

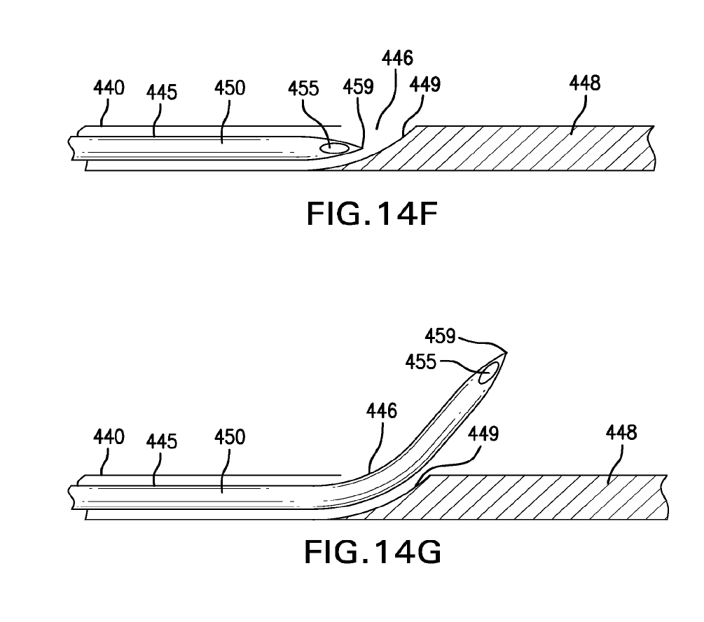

FIGS. 14A-14H show one embodiment of a delivery catheter 400.

FIGS. 14A-14B show side and end views of delivery catheter 400. Delivery catheter 400 includes a balloon 410, a proximal cap 420, a distal cap 430, a plurality of needle housings 440, and a plurality of delivery needles 450.

FIG. 14C shows another end view of delivery catheter 400. Delivery catheter 400 includes a needle lumen 405 and an inflation lumen 406. Delivery catheter may also include one or more steering lumens 407 and a guidewire lumen 408.

FIG. 14D shows an assembly view of delivery catheter 400. Balloon 410 includes a proximal portion 412 and a distal portion 414. Proximal cap 420 is coupled to proximal portion 412 of balloon 410. Distal cap 430 is slidably coupled to distal portion 414 of balloon 410. Distal portion 414 of balloon 410 may include a stop 413 which prevents distal cap 430 from sliding off. Needle housings 440 have a substantially helical configuration. Each needle housing 440 includes a proximal portion 442 and a distal portion 444. Proximal portions 442 of needle housings 440 are coupled to proximal cap 420. Distal portions 444 of needle housings 440 are coupled to distal cap 430. Each needle housing 440 includes a needle lumen 445. A delivery needle 450 is slidably disposed within each needle lumen 445. Delivery needles 450 may be coupled to a manifold 456 which distributes an agent to delivery needles 450.

FIG. 14E shows an enlarged view of distal cap 430. Distal cap 430 freely slides along and rotates around distal portion 414 of balloon 410.

FIGS. 14F-14G show enlarged views of needle housing 440. Needle housing 440 includes a needle lumen 445 formed proximally to a needle port 446. Needle lumen 445 is in communication with needle port 446. Needle port 446 is formed in an outwardly-facing surface of needle housing 440. Delivery needle 450 may be advanced and withdrawn through needle port 446. Needle lumen 445 may include a ramp 449 which directs delivery needle 450 out through needle port 446. Needle housing 440 may include an imaging marker 448. Imaging marker 448 may be a radioopaque material, coating, or other suitable marker for aiding visualization of needle housing 440. Delivery needle 450 includes a delivery lumen 455. Delivery needle 450 includes a tip 459 configured to penetrate the wall of a vessel. FIG. 14F shows needle housing 440 with delivery needle 450 retracted. FIG. 14G shows needle housing 440 with delivery needle 450 advanced through needle port 446.

Balloon 410 is sufficiently rigid to maintain the spacing between proximal cap 420 and distal cap 430, yet flexible enough to bend 90 degrees or more. Like balloon 410, needle housings 440 are also flexible enough to bend 90 degrees or more, which allows delivery catheter 400 to navigate into branched vessels, such as from the aorta into the renal arteries.

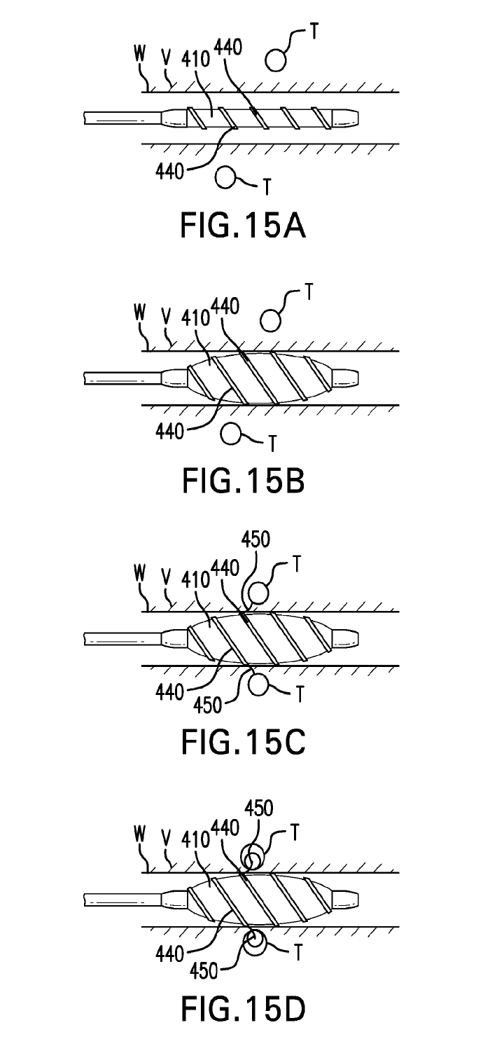

FIGS. 15A-15D show one embodiment of a method for using delivery catheter 400. FIG. 15A shows delivery catheter 400 advanced into a vessel V and balloon 410 positioned at or near one or more target sites T. FIG. 15B shows balloon 410 expanded and needle housings 440 brought into contact with walls W of vessel V. FIG. 15C shows delivery needles 450 advanced out of needle housings 440 and into the walls W. FIG. 15D shows delivery needles 450 delivering one or more agents to the target sites T. After delivery is complete, needles 450 are retracted back into needle housings 440 and balloon 410 deflated.

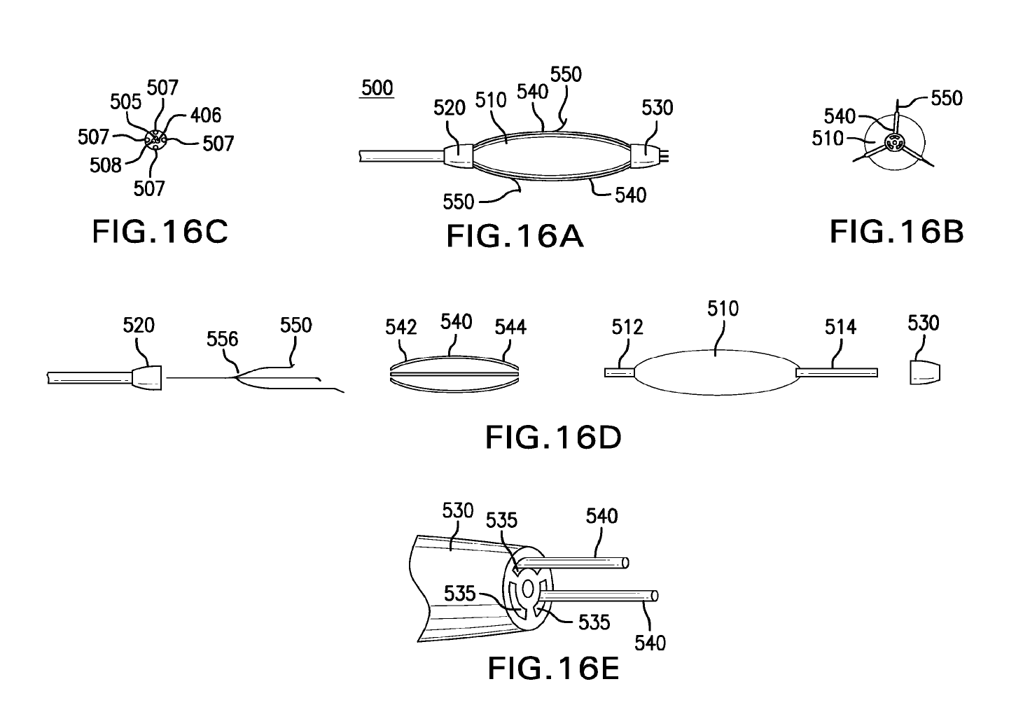

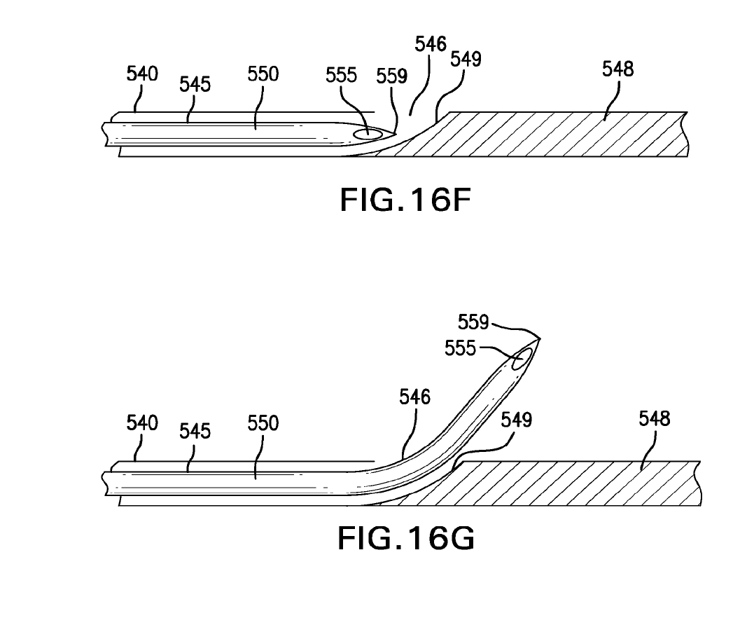

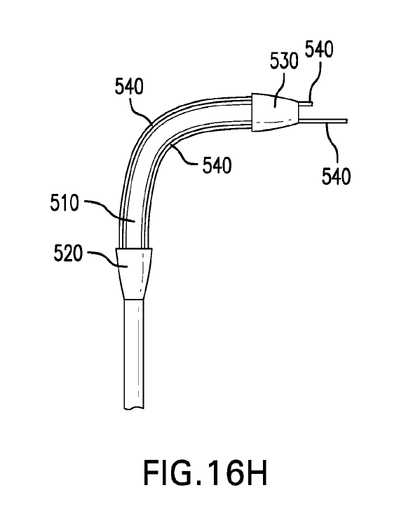

FIGS. 16A-16H show another embodiment of a delivery catheter 500.

FIGS. 16A-16B show side and end views of delivery catheter 500. Delivery catheter 500 includes a balloon 510, a proximal cap 520, a distal cap 530, a plurality of needle housings 540, and a plurality of delivery needles 550.

FIG. 16C shows another end view of delivery catheter 500. Delivery catheter 500 includes a needle lumen 505 and an inflation lumen 506. Delivery catheter may also include one or more steering lumens 507 and a guidewire lumen 508.

FIG. 16D shows an assembly view of delivery catheter 500. Balloon 510 includes a proximal portion 512 and a distal portion 514. Proximal cap 520 is coupled to proximal portion 512 of balloon 510. Distal cap 530 is coupled to distal portion 514 of balloon 510. Each needle housing 540 includes a proximal portion 542 and a distal portion 544. Proximal portions 542 of needle housings 540 are fixedly coupled to proximal cap 520. Distal portions 544 of needle housings 540 slide freely through distal cap 530. Each needle housing 540 includes a needle lumen 545. A delivery needle 550 is slidably disposed within each needle lumen 545. Delivery needles 550 may be coupled to a manifold 556 which distributes an agent to delivery needles 550.

FIG. 16E shows an enlarged view of distal cap 530. Distal cap 530 includes one or more openings 535 through which needle housings 540 may slide freely.

FIGS. 16F-16G show enlarged views of needle housing 540. Needle housing 540 includes a needle lumen 545 formed proximally to a needle port 546. Needle lumen 545 is in communication with needle port 546. Needle port 546 is formed in an outwardly-facing surface of needle housing 540. Delivery needle 550 may be advanced and withdrawn through needle port 546. Needle lumen 545 may include a ramp 549 which directs delivery needle 550 out through needle port 546. Needle housing 540 may include an imaging marker 548. Imaging marker 548 may be a radioopaque material, coating, or other suitable marker for aiding visualization of needle housing 540. Delivery needle 550 includes a delivery lumen 555. Delivery needle 550 includes a tip 559 configured to penetrate the wall of a vessel. FIG. 16F shows needle housing 540 with delivery needle 550 retracted. FIG. 16G shows needle housing 540 with delivery needle 550 advanced through needle port 546.

FIG. 16H shows delivery catheter 500 being bent at a 90 degree angle. Balloon 510 is sufficiently rigid to maintain the spacing between proximal cap 520 and distal cap 530, yet flexible enough to bend 90 degrees or more. Like balloon 510, needle housings 540 are also flexible enough to bend 90 degrees or more, which allows delivery catheter 500 to navigate into branched vessels, such as from the aorta into the renal arteries. Needle housings 540 slide freely through distal cap 530, which allows a needle housing 540 on the inside of a bend to slide further through distal cap 530, while allowing a needle housing 540 on the outside of a bend to slide not as far through distal cap 530. Distal cap 530 may be of sufficient length or otherwise configured to prevent distal portion 544 of needle housing 540 from sliding completely out of distal cap 530.

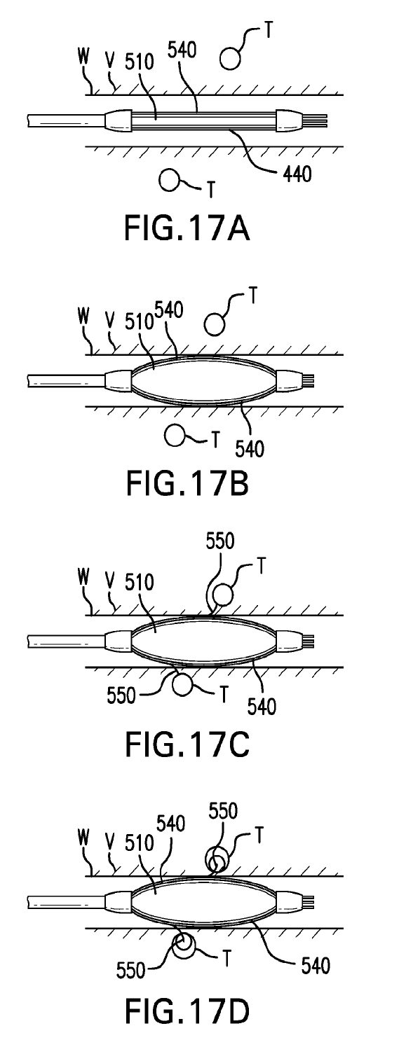

FIGS. 17A-17D show one embodiment of a method for using delivery catheter 500. FIG. 17A shows delivery catheter 500 advanced into a vessel V and balloon 510 positioned at or near one or more target sites T. FIG. 17B shows balloon 510 expanded and needle housings 540 brought into contact with walls W of vessel V. FIG. 17C shows delivery needles 550 advanced out of needle housings 540 and into the walls W. FIG. 17D shows delivery needles 550 delivering one or more agents to the target sites T. After delivery is complete, needles 550 are retracted back into needle housings 540 and balloon 510 deflated.

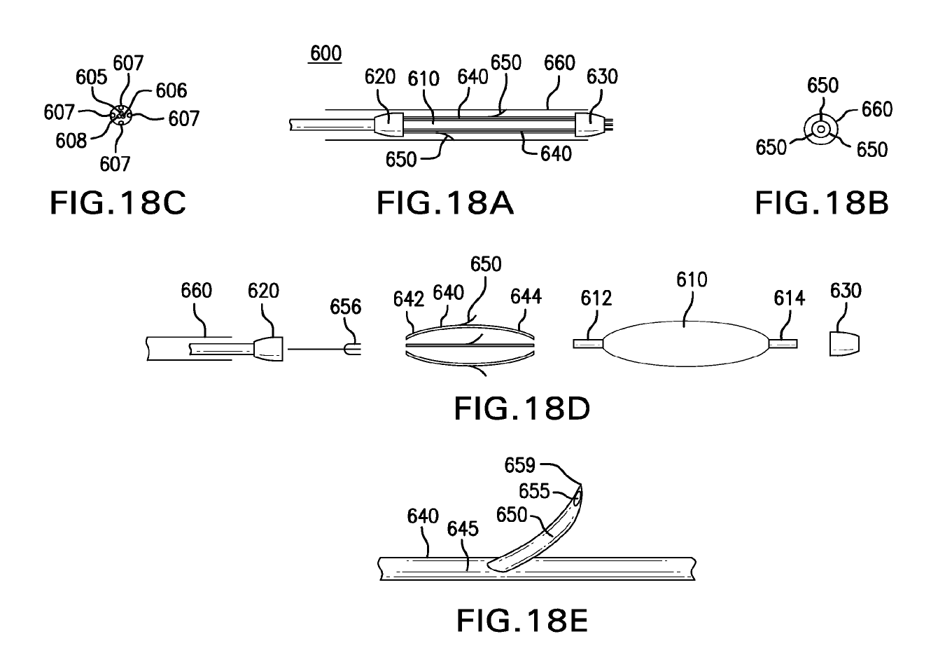

FIGS. 18A-18E show yet another embodiment of a delivery catheter 600.

FIGS. 18A-18B show side and end views of delivery catheter 600. Delivery catheter 600 includes a balloon 610, a proximal cap 620, a distal cap 630, a plurality of needle supports 640, a plurality of delivery needles 650, and a sheath 660.

FIG. 18C shows another end view of delivery catheter 600. Delivery catheter 600 includes a needle lumen 605 and an inflation lumen 606. Delivery catheter may also include one or more steering lumens 607 and a guidewire lumen 608.

FIG. 18D shows an assembly view of delivery catheter 600. Balloon 610 includes a proximal portion 612 and a distal portion 614. Proximal cap 620 is coupled to proximal portion 612 of balloon 610. Distal cap 630 is coupled to distal portion 614 of balloon 610. Each needle support 640 includes a proximal portion 642 and a distal portion 644. Proximal portions 642 of needle supports 640 are coupled to proximal cap 620. Distal portions 644 of needle supports 640 are coupled to distal cap 630. Each needle support 640 includes a delivery lumen 645. A delivery needle 650 is coupled to a side of each needle support 640 in fluid communication with delivery lumen 645. Delivery needles 650 are outwardly biased, and may be constrained or deployed by sheath 660 slidably positioned around delivery needles 650. Needle supports 640 may be coupled to a manifold 656 which distributes an agent to delivery lumens 645.

FIG. 18E shows an enlarged view of needle support 640 and delivery needle 650. Needle support 640 includes a delivery lumen 645 formed proximally to delivery needle 650. Delivery needle 650 includes a delivery lumen 655. Delivery lumen 645 of needle support 640 is in fluid communication with delivery lumen 655 of needle 650. Delivery needle 650 includes a tip 659 configured to penetrate the wall of a vessel. Needle support 640 may include an imaging marker 648. Imaging marker 648 may be a radioopaque material, coating, or other suitable marker for aiding visualization of needle support 640.

Balloon 610 is sufficiently rigid to maintain the spacing between proximal cap 620 and distal cap 630, yet flexible enough to bend 90 degrees or more. Like balloon 610, needle supports 640 are also flexible enough to bend 90 degrees or more, which allows delivery catheter 600 to navigate into branched vessels, such as from the aorta into the renal arteries.

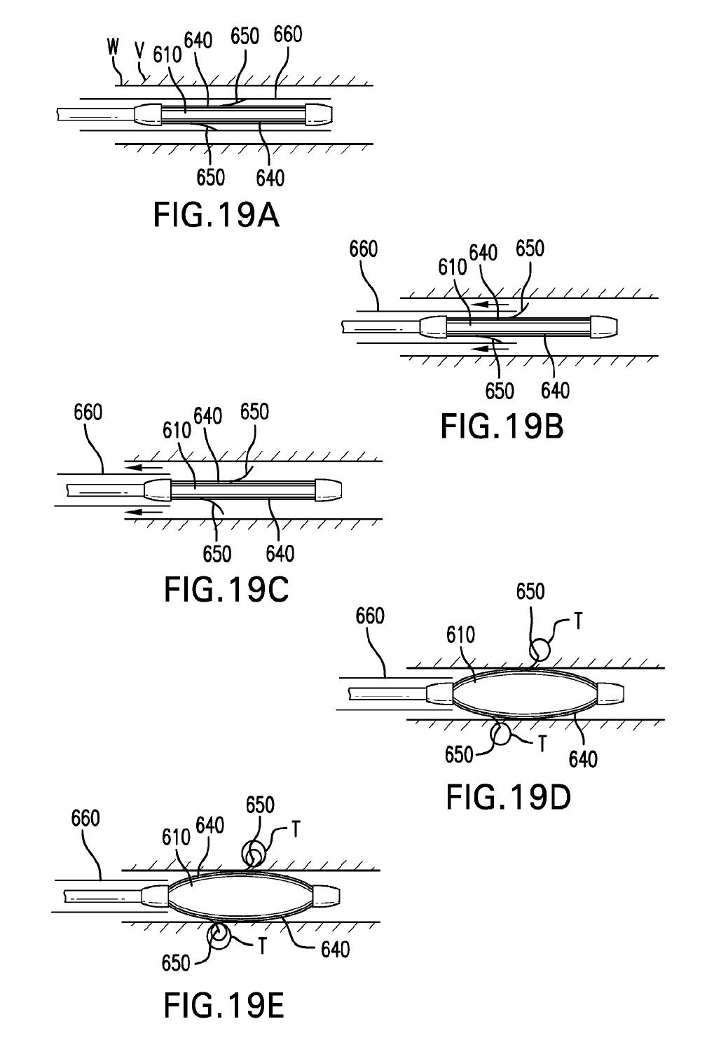

FIGS. 19A-19E show one embodiment of a method for using delivery catheter 600. FIG. 19A shows delivery catheter 600 advanced into a vessel V and balloon 610 positioned at or near one or more target sites T. FIG. 18B shows sheath 660 partially retracted from delivery needles 650. FIG. 18C shows sheath 660 completely retracted from delivery needles 650, with delivery needles 650 pointing outwards. FIG. 18D shows balloon 610 expanded and delivery needles 650 forced into the walls W. FIG. 18E shows delivery needles 650 delivering one or more agents to the target sites T. After delivery is complete, balloon 610 is deflated and sheath 660 is advanced back over needles 650.

Delivery catheters 400, 500, and 600 are capable of injecting small volumes of agents, 0.005-0.5 ml, or 0.05-0.3 ml per injection site (or 0.05-3 ml total volume, or 0.5-1 ml total volume) to very localized sites within the body. These delivery catheters are capable of specifically targeting nerve cells and portions of the nerve cell, and locally affecting nerve function and provide therapeutic benefit from a degenerated and overactive sympathetic nervous system. Such low volumes reduce loss of agent into the systemic circulation and reduce damage to surrounding tissue and organs.

By contrast, tissue damage zones induced by radiofrequency ablation and guanethidine-induced denervation are quite macroscopic. RF ablation requires the creation of five to eight lesions along the renal artery; typical dimensions range between 2-3 mm in size. About 6 ml of guanethidine is injected into the vessel wall causing a large, single damage zone of about 10 mm. In addition, there may be significant pain associated with the RF ablation clinical procedure; patients are often sedated during ablation. The delivery catheters described above reduce tissue damage and pain during the procedure by precisely delivering microvolumes of agent per injection site without the need for sedation during a procedure.

Delivery catheters 400, 500, and 600 are: (i) sufficiently flexible to access the target site (the catheter is sufficiently flexible to access the renal arteries), (ii) small in profile, to minimize injury during introduction and delivery, (iii) configured to provide perfusion during agent delivery, (iv) constructed of materials which enhance visibility under fluoroscopy to help accurately position the device and deliver the agents to precise locations within the tissue, and (v) configured with needles of suitable quantity, locations, and depths for delivery and distribution of an agent to targeted sites (an anatomic location in a body, targeted sites within tissue, targeted sites in a nerve cell bundle, and targeted sites within nerve cells), while reducing systemic losses into the circulation and reducing collateral tissue or organ damage.

Balloons 410, 510, and 610 may be positioning component which help to hold delivery catheters 400, 500, and 600 in place and assist with the advancement of delivery needles 450, 550, and 650 through the vessel wall W to nerve cell bundles in the adventitia. Balloons 410, 510, and 610 may be made of compliant materials such as nylon or polyurethane. Balloons 410, 510, and 610 may expand at very low pressures, such as approximately 1-2 atmospheres, to prevent injury to the vessel wall W.

Delivery catheters 400, 500, and 600 may be configured to provide blood perfusion during the procedure. The size, number, and shape of needle housings 440 and 540, and needle supports 640, may be configured so that balloons 410, 510, and 610 do not contact the vessel wall W, and vessel wall contact is limited to needle housings 440 and 540, and needle supports 640, only. Balloons 410, 510, and 610 position delivery catheters 400, 500, and 600, assists in conforming needle housings 440, 540, and 640 to the vessel wall W, and helps advance delivery needles 450, 550, and 650 to the targeted sites.

Delivery needles 450, 550, and 650 may be made of Nitinol, stainless steel, or Elgiloy for sufficient stiffness and strength to penetrate the vessel wall W. Delivery needles 450, 550, and 650 may be coated with radioopaque coatings of gold, platinum or platinum-iridium alloy, tantalum, or tungsten to improve the visibility and visualize the advancement of delivery needles 450, 550, and 650 under fluoroscopy.

Delivery needles 450, 550, and 650 may be made of magnetic materials with a very high magnetic permeability such that they are responsive to an external stimulus in a magnetic field. Examples of magnetic materials include, carbon steels, nickel and cobalt-based alloys, Alnico (a combination of aluminum, nickel and cobalt), Hyperco alloy, neodymium-iron boron and samarium-cobalt. Delivery needles 450, 550, and 650 may be advanced into the vessel wall W in a magnetic field using external computer-controlled console systems, such as those manufactured by Stereotaxis. Externally guided ultrasound systems using sound waves traveling through blood may be used to assist with the precise penetration of delivery needles 450, 550, and 650 into the vessel wall W. Delivery needles 450, 550, and 650 may be operated using intravascular microelectromechanical systems (MEMS) that may advance delivery needles 450, 550, and 650 into the vessel wall W using external and/or internal guidance.

Other imaging modalities may be integrated into delivery catheters 400, 500, and 600 to precisely locate target regions inside the body and locally deliver agents within the vessel wall W. These include intravascular ultrasound (IVUS) and optical coherence tomography (OCT) imaging, both of which, have capabilities to distinguish the different layers of the vessel wall (endothelium, intima, media and adventitia). Miniaturized IVUS and OCT sensors can be embedded along the shaft of delivery catheters 400, 500, and 600 and used to track the advancement of delivery needles 450, 550, and 650 into the adventitia. IVUS sensors send sound waves in the 20-40 MHz frequency range; the reflected sound waves from the vessel wall are received through an external computerized ultrasound equipment which reconstructs and displays a real-time ultrasound image of the blood vessel surrounding the sensor. Similarly, OCT sensors produce real-time, high resolution images of the vessel wall (on the order of microns) on computer displays using interferometric methods employing near-infrared light. Both sensors may be located on delivery catheters 400, 500, and 600 near needle ports 446 and 546 at the proximal, middle, or distal segments of balloons 410, 510, and 610. Once the position of delivery needles 450, 550, and 650 is verified, the agent is delivered and delivery needles 450 and 550 retracted.

The description and examples given above describe affecting the function of nerves surrounding the renal arteries to control hypertension. However, the described devices, methods, agents, and delivery methods may be used to treat other diseases through local delivery of agents to affect nerve function at various locations along the sympathetic nervous system in the human body. These include and are not limited to diabetes, tingling, tinnitus, fibromyalgia, impulse-control disorders, sleep disorders, pain disorders, pain management, congestive heart failure, sleep apnea, chronic kidney disease, and obesity. Other potential target sites and disease states are listed below.

| | Target location in the |
|---|---|
| Disease state or condition | sympathetic nervous system |
| Pulmonary hypertension, | Vagus nerve |
| arrhythmias, chronic hunger | |
| Pancreatitis, hepatitis, | Celiac ganglia (renal and |
| chronic kidney disease | adrenal nerves etc.) |
| Adrenal function, hypertension | Celiac ganglia, greater splanchnic nerve |
| Bladder incontinence | Pelvic nerve |
| Hypertension, glaucoma | Carotid artery and plexus |
| Sciatica | Sciatic nerve |
| Chicken pox, shingles | Dorsal root ganglia |
| Mood alteration | Vagus nerve, submaxillary, and |
| | sphenopalatine ganglia |

While the foregoing has been with reference to particular embodiments of the invention, it will be appreciated by those skilled in the art that changes in these embodiments may be made without departing from the principles and spirit of the invention.

What is claimed is:

1. A method for treating hypertension in a patient, the method comprising:
delivering a cardiac glycoside locally to a portion of a renal nerve in an amount sufficient to impair function of the renal nerve and lower a blood pressure of the patient.

2. The method of claim 1, wherein the amount of the cardiac glycoside delivered is sufficient to reduce a nerve conductance in the portion of the renal nerve.

3. The method of claim 1, wherein the amount of the cardiac glycoside delivered is sufficient to induce death of nerve cells in the portion of the renal nerve.

4. The method of claim 1, wherein the amount of the cardiac glycoside delivered is sufficient to induce death of nerve cells in the portion of the renal nerve and prevent regrowth of nerve cells.

5. The method of claim 1, wherein the amount of the cardiac glycoside delivered is sufficient to impair nerve function by acting on an axonal segment of the nerve cells in the portion of the renal nerve.

6. The method of claim 1, wherein the amount of the cardiac glycoside delivered is sufficient to impair nerve function by inducing neuro-muscular block, sensory nerve block, or clinical nerve block.

7. The method of claim 1, wherein the amount of the cardiac glycoside delivered does not cause damage to tissue surrounding the renal nerve.

8. The method of claim 1, wherein function of the renal nerve is impaired temporarily.

9. The method of claim 1, wherein function of the renal nerve is impaired for a sustained period of time.

10. The method of claim 1, wherein the cardiac glycoside is delivered in a time release formulation.

11. The method of claim 1, wherein the cardiac glycoside is digoxin.

12. The method of claim 1, wherein the amount of the cardiac glycoside delivered is approximately 0.2-1 mg/kg.

13. The method of claim 1, wherein the volume of the cardiac glycoside delivered is approximately 0.05-5 cc per administration.

14. The method of claim 1, wherein the amount of cardiac glycoside delivered is small enough and does not substantially enter the systemic circulation or cause organ damage.

15. The method of claim 1, wherein the amount of the cardiac glycoside delivered is sufficient to impair nerve function by acting on Schwann cells.

16. A method for treating hypertension in a patient, the method comprising:
delivering a mixture of a cardiac glycoside, an ACE inhibitor, and an NSAID locally to a portion of a renal nerve in an amount sufficient to impair function of the renal nerve and lower a blood pressure of the patient.

17. The method of claim 16, wherein the amount of the mixture delivered is sufficient to reduce a nerve conductance in the portion of the renal nerve.

18. The method of claim 16, wherein the amount of the mixture delivered is sufficient to induce death of nerve cells in the portion of the renal nerve.

19. The method of claim 16, wherein the amount of the mixture delivered is sufficient to induce death of nerve cells in the portion of the renal nerve and prevent regrowth of nerve cells.

20. The method of claim 16, wherein the amount of the mixture delivered does not cause damage to tissue surrounding the renal nerve.

21. The method of claim 16, wherein function of the renal nerve is impaired temporarily.

22. The method of claim 16, wherein function of the renal nerve is impaired for a sustained period of time.

23. The method of claim 16, wherein the mixture is delivered in a time release formulation.

24. The method of claim 16, wherein the cardiac glycoside is digoxin.

25. The method of claim 16, wherein the ACE inhibitor is captopril.

26. The method of claim 16, wherein the non-steroidal anti-inflammatory is indomethacin.

27. The method of claim 16, wherein the amount of the mixture delivered is approximately 0.2-2 mg/kg of the cardiac glycoside, approximately 2-20 mg/kg of the ACE inhibitor, and approximately 0.2-2 mg/kg of the NSAID.

28. A method for treating a disease condition of the autonomic nervous system in a patient, the method comprising:
delivering an agent to a portion of a targeted nerve in an amount sufficient to affect function of the targeted nerve and alleviate one or more symptoms of the disease condition in the patient.

29. The method of claim 28, wherein the condition is hypertension, and the symptoms include high blood pressure.

30. The method of claim 28, wherein the condition is asthma, and the symptoms include difficulty in breathing.

31. The method of claim 28, wherein the condition is depression, fibromyalgia, dementia, attention deficit hyperactivity disorder and migraine headaches, and the symptoms include decreased attention, discomfort and overstimulation. congestive heart failure, and the symptoms include shortness of breath, leg swelling, and the inability of the heart to pump sufficient blood into the circulatory system.

32. The method of claim 28, wherein the condition is obesity, and the symptoms include uncontrolled weight gain.

33. The method of claim 28, wherein the condition is atrial fibrillation, and the symptoms include heart palpitations, dizziness, lack of energy and chest discomfort.

34. The method of claim 28, wherein the agent is a cardiac glycoside.

35. The method of claim 34, wherein the cardiac glycoside is digoxin.

36. The method of claim 28, wherein the agent is an ion channel blocker.

37. The method of claim 36, wherein the ion channel blocker is phenytoin.

38. The method of claim 36, wherein the ion channel blocker is carbamazepine or lithium chloride.

39. The method of claim 28, wherein the agent is an ACE inhibitor.

40. The method of claim 28, wherein the agent is an antibiotic.

41. The method of claim 28, wherein the agent is a excitatory glutamate receptor.

42. The method of claim 28, wherein the agent includes two or more constituents.

43. The method of claim 28, wherein the agent is a mixture of a cardiac glycoside, an ACE inhibitor, and an NSAID.

44. The method of claim 28, wherein the portion of the targeted nerve is located in the wall of a blood vessel.

45. The method of claim 28, wherein the targeted nerve is a renal nerve.

46. The method of claim 28, wherein the agent is delivered locally.

47. The method of claim 28, wherein the agent is delivered orally.

48. The method of claim 28, wherein the targeted nerve is affected by temporary or sustained neuro-muscular block.

49. The method of claim 28, wherein the targeted nerve is affected by sensory nerve block or clinical nerve block.

50. The method of claim 28, wherein the targeted nerve is affected by reduced or blocked nerve conductance.

51. The method of claim 28, wherein the targeted nerve is affected by nerve cell death.

52. The method of claim 28, wherein the targeted nerve is affected by damage to axonal segments of neurons.

53. The method of claim 28, wherein the agents are selected from one or more of the following: agents which inhibit sodium-potassium pumps, calcium channels and sodium channels in nerve cells; angiotensin converting enzymes; glutamate receptors; COX-1 and COX-2 receptors in nerve cells.

54. The method of claim 28, wherein the amount of agent delivered is small enough and does not substantially enter the systemic circulation or cause organ damage.

55. The method of claim 28, wherein the amount of agent delivered is sufficient to impair nerve function by acting on Schwann cells.

56. The method of claim 28, wherein the therapy is delivered with minimal pain during the clinical procedure without the use of sedatives.

57. A delivery catheter comprising:
a balloon having a proximal portion and a distal portion;
a proximal cap coupled to the proximal portion of the balloon;
a distal cap slidably coupled to the distal portion of the balloon;
a plurality of needle housings having proximal portions and distal portions, the proximal portions of the needle housings being coupled to the proximal cap, the distal portions of the needle housings being coupled to the distal cap, the needle housing having a substantially helical configuration; and
a delivery needle slidably disposed within a needle lumen formed in each of the needle housings, the delivery needles capable of being advanced and retracted through a needle port formed in an outwardly-facing side of each needle housing.

58. A delivery catheter comprising:
a balloon having a proximal portion and a distal portion;
a proximal cap coupled to the proximal portion of the balloon;
a distal cap coupled to the distal portion of the balloon;
a plurality of needle housings having proximal portions and distal portions, the proximal portions of the needle housings being coupled to the proximal cap, the distal portions of the needle housings being slidably disposed within one or more openings in the distal cap; and
a delivery needle slidably disposed within a needle lumen formed in each of the needle housings, the delivery needles capable of being advanced and retracted through a needle port formed in an outwardly-facing side of each needle housing.

59. A delivery catheter comprising:
a balloon having a proximal portion and a distal portion;
a proximal cap coupled to the proximal portion of the balloon;
a distal cap coupled to the distal portion of the balloon;
a plurality of needle supports having proximal portions and distal portions, the proximal portions of the needle supports being coupled to the proximal cap, the distal portions of the needle supports being coupled to the distal cap, each of the needle supports having a delivery lumen;
a delivery needle coupled to each needle support, the delivery needles being outwardly biased, each of the delivery needles having a delivery lumen in fluid communication with the delivery lumen of each needle support; and
a sheath slidably coupled around the delivery needles, the sheath capable of constraining the delivery needles.
